(19) United States
(12) Patent Application Publication
(10) Pub. No.: US 2022/0372522 A1
(43) Pub. Date: Nov. 24, 2022

(54) COMPOSITIONS AND METHODS FOR HOMOLOGY-DIRECTED RECOMBINATION

(71) Applicant: LIFE TECHNOLOGIES CORPORATION, Carlsbad, CA (US)

(72) Inventors: Xiquan LIANG, Escondido, CA (US); Pei-Zhong TANG, Carlsbad, CA (US); Robert POTTER, San Marcos, CA (US)

(21) Appl. No.: 17/766,998

(22) PCT Filed: Oct. 7, 2020
(86) PCT No.: PCT/US2020/054526

§ 371 Date: Apr. 6, 2022

Related U.S. Application Data
(60) Provisional application No. 62/912,115, filed on Oct. 8, 2019.

(51) Int. Cl. C12N 15/90; C12N 9/22; C12N 15/11; C07K 14/245

(52) U.S. Cl. CPC C12N 15/90; C12N 9/22; C12N 15/11; C12N 15/111; C07K 14/245; C12N 2800/80; C12N 2310/20

(57) ABSTRACT

The present disclosure relates, in part, to improved methods of making single-stranded DNA (ssDNA) from double-stranded DNA (dsDNA), as well as use of the resulting ssDNA for genome engineering. The disclosure also relates, in part, to improved methods of genetic modification using single stranded DNA binding proteins.

### SEQUENCE LISTING

The instant application contains a Sequence Listing which has been submitted electronically in ASCII format and is hereby incorporated by reference in its entirety. Said ASCII copy, created on Sep. 30, 2020, is named LT01478PCT_SL.txt and is 54,080 bytes in size.

### FIELD OF THE INVENTION

The present disclosure relates, in part, to improved methods of making single-stranded DNA (ssDNA) from double-stranded DNA (dsDNA), as well as use of the resulting ssDNA for genome engineering. The disclosure also relates to improved methods of genetic modification, in some aspects, using single stranded DNA binding proteins.

### BACKGROUND

The recent advances in TALENs or CRISPR-mediated genome editing tools enable researchers to introduce double-strand breaks (DSBs) in mammalian genome efficiently. The DSBs are then mostly repaired by either the non-homologous end joining (NHEJ) pathway or the homology-directed repair (HDR) pathway. Genome editing efficiencies can vary widely, depending on the cell type, mode of introduction of genome editing reagents into the cells, and the like. In mammalian cells, the NHEJ pathway is predominant and error-prone. However, the HDR pathway allows for precise genome editing via the use of sister chromatids or exogenous DNA molecules. Many attempts have been made to improve the HDR efficiency, but the efficiency remains relatively low.

Single stranded DNA (ssDNA) can be a good substrate for homologous recombination, because it is not easily randomly recombined into the genome. However, large ssDNA is difficult to synthesize. Exonucleases that digest just one strand of a double stranded DNA molecule (e.g., lambda exonuclease) often stall during digestion, resulting in partial digestion.

There remains a need for improved methods for genome editing (i.e., either NHEJ-mediated and HDR-mediated genome editing), as well as improved methods for making ssDNA for HDR.

### SUMMARY

The instant technology generally relates to methods, compositions, and kits for improving editing efficiency of genome editing tools. As discussed herein, the embodiments disclosed herein are based, in part, on the surprising discovery that addition of a non-specific DNA binding protein (e.g., a DNA binding protein that does not target a specific sequence, such as single stranded binding protein) to a genome editing reaction increased the efficiency of both NHEJ-mediated and HDR-mediated genome editing. This technology further relates to improved methods for making single-stranded DNA (ssDNA) from double-stranded DNA (dsDNA) templates.

In one aspect, a method for preparing single stranded DNA (ssDNA) is provided. In embodiments, the method includes (a) providing a composition comprising a double stranded DNA (dsDNA); (b) contacting the composition with a first exonuclease (e.g., a 5′ to 3′ exonuclease such as lambda exonuclease, or the like); and (c) contacting the composition comprising the first exonuclease with a second exonuclease (e.g., a 3′ to 5′ exonuclease such as exonuclease III, or the like or a 5′ to 3′ exonuclease such as T7 exonuclease, or the like). In embodiments, step (b) and step (c) are performed without changing buffer (e.g., reaction buffer). In embodiments, step (c) is performed without inactivating the lambda exonuclease.

In embodiments, the dsDNA has a modification on the 5′ end of one strand. In embodiments, the 5′ modification is a 5′ phosphate. In embodiments, the first exonuclease can be a 5′ to 3′ exonuclease that only digests the strand having the modification. In embodiments, the first exonuclease is a 5′ to 3′ exonuclease that only digests the strand without the modification.

In embodiments, the second exonuclease is an exonuclease, e.g. 3′ to 5′ exonuclease, that only digests a blunt end of dsDNA, i.e., that does not have any overhang.

In embodiments, the composition includes a detectable marker that binds to a nucleotide. In embodiments, the detectable marker preferentially interacts with ssDNA. In embodiments, the detectable marker preferentially interacts with dsDNA. In embodiments, the detectable marker is a fluorescent nucleic acid stain. In embodiments, the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium (PICOGREEN®).

In embodiments, the method includes monitoring an amount of detectable marker. In embodiments, the amount of detectable marker is monitored in real time. In embodiments, the amount of detectable marker is monitored by QUBIT™ Fluorometric Quantification system (Thermo Fisher Scientific, Waltham, Mass.), or similar system.

In embodiments, the second exonuclease is added when the amount of ssDNA reaches a plateau. In embodiments, the second exonuclease is added when the amount of dsDNA reaches a plateau. In embodiments, the second exonuclease is added when the amount of the detectable marker reaches a plateau.

In embodiments, the reaction is stopped when the amount of ssDNA reaches a plateau. In embodiments, the reaction is stopped when the amount of dsDNA reaches a plateau. In embodiments, the reaction is stopped when the amount of the detectable marker reaches a plateau.

In embodiments, the temperature of the composition does not exceed about 50° C. In embodiments, the temperature of the composition is maintained between about 20° C. and about 50° C. In embodiments, the temperature of the composition is maintained between about 30° C. and about 40° C. In embodiments, the temperature of the composition is maintained at about 37° C. In embodiments, the temperature of the composition is maintained at about room temperature. In embodiments, the temperature is maintained at the indicated temperature/range throughout the method.

In embodiments, step (b) is performed for a period of time sufficient for at least partial digestion of a strand of the dsDNA. In embodiments, step (c) is performed for a period of time sufficient for at least partial digestion of a strand of the dsDNA. The amount of time required for digestion depends on the length of the dsDNA and/or the type of exonuclease used.

In embodiments, the method includes (d) adding a stop buffer.

In embodiments, the ssDNA is purified after step (c) or (d).

In embodiments, the dsDNA is between 100 and 10,000 base pairs in length.

In embodiments, the composition includes magnesium. In embodiments, the composition includes between 1 mM and 10 mM MgCl₂.

In an aspect, a composition including dsDNA, a first exonuclease, such as a 5′ to 3′ exonuclease (e.g., lambda exonuclease), a second exonuclease, such as a 3′ to 5′ exonuclease (e.g., exonuclease III), and a detectable marker that binds to nucleotides is provided. In embodiments, the composition includes magnesium. In embodiments, the composition includes between 1 mM and 10 mM MgCl₂.

In an aspect, a kit including a first exonuclease, such as a 5′ to 3′ exonuclease (e.g., lambda exonuclease), a second exonuclease, such as a 3′ to 5′ exonuclease (e.g., exonuclease III), and a detectable marker that binds to nucleotides is provided. In embodiments, the kit includes a magnesium salt.

In an aspect, a system including (a) a device configured to detect a detectable marker that binds to a nucleotide; and (b) a composition comprising DNA, an exonuclease, and a detectable marker that binds to nucleotides. In embodiments, the exonuclease is a lambda exonuclease and/or exonuclease III. In embodiments, the exonuclease is a lambda exonuclease and exonuclease III.

In embodiments, the detectable marker is a fluorescent nucleic acid stain. In embodiments, the detectable marker preferentially interacts with ssDNA. In embodiments, the detectable marker preferentially interacts with dsDNA. In embodiments, the detectable marker is a fluorescent nucleic acid stain. In embodiments, the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium (PICOGREEN®).

In an aspect, a method for preparing a single stranded target DNA (ssDNA) is provided. In embodiments, the method includes (a) providing a template DNA comprising a target donor DNA sequence; (b) providing an amplification primer pair comprising a forward primer and a reverse primer designed to amplify the target donor DNA sequence, wherein the forward primer comprises a 5′ end comprising ribonucleotides and a 3′ end comprising deoxynucleotides, and wherein the reverse primer is not susceptible to digestion by RNase H; (c) amplifying the target donor DNA sequence with the forward and reverse primers to generate an amplification product comprising a first strand and a second strand complementary to the first strand, wherein the 5′ end of the first strand of the amplification product is susceptible to digestion by RNaseH; (d) contacting the amplification product with an RNase H exonuclease, and; (e) contacting the amplification product with a second exonuclease that is a 5′ to 3′ exonuclease. In embodiments, steps (c) and (d) are simultaneous. In embodiments, steps (c) and (d) are sequential.

In embodiments, the method includes contacting the amplification product with a third exonuclease.

In embodiments, the second exonuclease is Lambda exonuclease. In embodiments, the third exonuclease is a 3′ to 5′ exonuclease. In embodiments, the third exonuclease is Exonuclease III.

In embodiments, the amplification product is contacted with the RNaseH, second exonuclease, and third exonuclease simultaneously.

In embodiments, the amplification product is generated by polymerase chain reaction.

In an aspect a method for genetically modifying a cell is provided. In some embodiments, provided is a method of NHEJ-mediated genetic modification at a predetermined locus in a cell. In embodiments, the method includes introducing into the cell: (i) at least one single stranded DNA binding protein or nucleic acid encoding the at least one single stranded DNA binding protein; and (ii) at least one nucleic acid cutting entity or nucleic acid encoding the at least one nucleic acid cutting entity; under conditions that allow for genetically modifying the cell at a predetermined locus.

In other embodiments, provided is a method of HDR-mediated genetic modification at a predetermined locus in a cell is provided. In such embodiments, the method includes introducing into the cell: (i) at least one donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity. In some embodiments, the method is performed under conditions that allow for genetically modifying the cell at a predetermined locus. In some embodiments, the donor DNA is single stranded. In some embodiments, the donor DNA is double stranded.

In an aspect a method for improving targeting efficiency of a nucleic acid cutting entity for genetic modification of a cell is provided. In embodiments, the method includes introducing into the cell: (i) at least one single stranded DNA binding protein or nucleic acid encoding the at least one single stranded DNA binding protein; and (ii) at least one nucleic acid cutting entity or nucleic acid encoding the at least one nucleic acid cutting entity; under conditions that allow for genetically modifying the cell at a predetermined locus. In some embodiments, the genetic modification is NHEJ-mediated genome editing. In some embodiments, the genetic modification is HDR-mediated genome editing, and the method further includes (iii) introducing into the cell at least one donor DNA molecule. In some embodiments, the donor DNA is single stranded. In some embodiments, the donor DNA is double stranded.

In an aspect a method for reducing off-target integration of a donor DNA during HDR-mediated genetic modification of a cell is provided. In embodiments, the method includes introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for HDR-mediated genetic modification of the cell at a predetermined locus.

In an aspect a method for enhancing delivery of a donor DNA to a cell for HDR-mediated genetic modification of the cell is provided. In embodiments, the method includes introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for HDR-mediated genetic modification of the cell at a predetermined locus.

In an aspect a method for reducing degradation of a donor DNA for genetic modification of a cell, comprising introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

In an aspect a method for genetically modifying a cell is provided. In embodiments, the method includes introducing into the cell at least one non-specific single strand DNA binding protein or nucleic acid encoding the at least one non-specific single stranded DNA binding protein in the presence of at least one nucleic acid cutting entity or nucleic acid encoding the at least one nucleic acid cutting entity; under conditions that allow for genetically modifying the cell at a predetermined locus. In some embodiments, the genetic modification is NHEJ-mediated genome editing. In some embodiments, the genetic modification is HDR-mediated genome editing, and the method further includes introducing a donor DNA into the cell under the conditions that allow for genetically modifying the cell at the predetermined locus.

In embodiments, one or more of the at least one nucleic acid cutting entity is a zinc finger nuclease; a TAL effector nuclease; and/or a CRISPR complex. In embodiments, the CRISPR complex is a Cas9/gRNA complex.

In embodiments that provide HDR-mediated genetic modification, and include the step of introducing into the cell at least one donor DNA molecule, the donor DNA molecule is contacted with the at least one single stranded DNA binding protein prior to introduction into the cell.

In embodiments, the at least one nucleic acid cutting entity or nucleic acid encoding the at least one nucleic acid cutting entity is introduced into the cell before introduction at least one non-specific single strand DNA binding protein or nucleic acid encoding the at least one non-specific single stranded DNA binding protein into the cell. In embodiments that include the step of introducing a donor DNA into the cell, the at least one nucleic acid cutting entity or nucleic acid encoding the at least one nucleic acid cutting entity is introduced into the cell before introduction of the donor DNA and/or at least one non-specific single strand DNA binding protein or nucleic acid encoding the at least one non-specific single stranded DNA binding protein into the cell.

In embodiments, the at least one nucleic acid cutting entity or nucleic acid encoding the at least one nucleic acid cutting entity is introduced into the cell after introduction of the at least one non-specific single strand DNA binding protein or nucleic acid encoding the at least one non-specific single stranded DNA binding protein into the cell. In embodiments that include the step of introducing a donor DNA into the cell, the at least one nucleic acid cutting entity or nucleic acid encoding the at least one nucleic acid cutting entity is introduced into the cell after introduction of the donor DNA and/or at least one non-specific single strand DNA binding protein or nucleic acid encoding the at least one non-specific single stranded DNA binding protein into the cell.

In embodiments, the non-specific DNA binding protein comprises an oligonucleotide/oligosaccharide-binding (OB)-fold. In embodiments, the non-specific DNA binding protein is SSB, RecA, or T4G32. In embodiments, the DNA binding protein is SSB or a variant thereof. In embodiments, the SSB is E. coli SSB or variant thereof. In embodiments, one or more of the at least one non-specific DNA binding protein comprises the E. coli SSB C-terminus or variant thereof. In embodiments, the E. coli SSB or variant thereof includes an N-terminal domain with multiple basic residues or positively charged amino acids, e.g. for DNA binding. In embodiments, the E. coli SSB or variant thereof includes a C-terminal domain with multiple negatively charged or acidic amino acids, e.g. for interaction with other SSB binding proteins. In embodiments, the E. coli SSB or variant thereof binds to ssDNA every 30 to 73 nucleotides, depending on the salt concentration.

In embodiments that include introduction of a donor DNA into the cell, the DNA binding protein is present in an amount sufficient to protect the donor DNA from degradation.

In embodiments, the DNA binding protein is present in an amount sufficient to improve the targeting efficiency of the at least one nucleic acid cutting entity.

In embodiments, the donor DNA comprises a nuclear localization signal (NLS). In embodiments, the DNA binding protein comprises a nuclear localization signal (NLS).

In embodiments, the single-strand DNA binding protein (and donor DNA, if present) are introduced into the cell using lipid transfection. In embodiments, single-strand DNA binding protein (and donor DNA, if present) are introduced into the cell using electroporation.

In embodiments that include introduction of donor DNA into the cell, the donor DNA is a single stranded DNA. In embodiments, the donor DNA is a double stranded DNA. In embodiments, the donor DNA is between 35 nucleotides and 10,000 nucleotides long.

In embodiments that include introduction of donor DNA into the cell, the donor DNA is associated with the DNA binding protein prior to introduction into the cell.

In an aspect, a composition including cells, \ at least one non-specific single strand DNA binding protein, and at least one nucleic acid cutting entity is provided. In some embodiments, the composition further comprises a donor DNA.

In embodiments, one or more of the at least one nucleic acid cutting entity is a zinc finger nuclease; a TAL effector nuclease; and/or a CRISPR complex. In embodiments, the CRISPR complex is a Cas9/gRNA complex.

In embodiments, one or more of the at least one non-specific DNA binding protein comprises a oligonucleotide/oligosaccharide-binding (OB)-fold. In embodiments, one or more of the at least one non-specific DNA binding protein is SSB, RecA, or T4G32. In embodiments, one or more of the at least one non-specific DNA binding protein is SSB or a variant thereof. In embodiments, the SSB is E. coli SSB or variant thereof. In embodiments, one or more of the at least one non-specific single strand DNA binding protein comprises the E. coli SSB C-terminus or variant thereof.

In embodiments, the donor DNA comprises a nuclear localization signal (NLS). In embodiments, the DNA binding protein comprises a nuclear localization signal (NLS).

In embodiments that include a donor DNA, the donor DNA is a single stranded DNA. In embodiments, the donor DNA is a double stranded DNA. In embodiments, the donor DNA is between 35 nucleotides and 10,000 nucleotides long.

In an aspect a kit for genetic modification is provided. In embodiments, the kit includes (i) a non-specific single strand DNA binding protein or nucleic acid encoding the at least one non-specific single stranded DNA binding protein; and (ii) a nucleic acid cutting entity or a nucleic acid encoding the nucleic acid cutting entity. In embodiments, the kit includes a lipid transfection reagent. In embodiments, the kit includes a non-homologous end joining (NHEJ) inhibitor.

In an aspect a method for preparing single stranded DNA (ssDNA) is provided. In embodiments, the method includes denaturing a double stranded DNA (dsDNA) in the presence of a single-strand DNA binding protein, thereby preparing ssDNA.

In embodiments, the single-strand DNA binding protein is SSB. In embodiments, the SSB is a thermostable SSB. In embodiments, the single strand DNA binding protein comprises the E. coli SSB C-terminus or variant thereof.

In embodiments, one strand of the dsDNA is labeled. In embodiments, the method includes isolating the labeled strand. In embodiments, the method includes depleting the labeled strand.

In an aspect, a method for genetically modifying a cell is provided. In embodiments, the method includes introducing into the cell the ssDNA made by a method described herein in the presence of a nucleic acid cutting entity or a nucleic acid encoding the nucleic acid cutting entity.

### BRIEF DESCRIPTION OF THE DRAWINGS

For a more complete understanding of the principles disclosed herein, and the advantages thereof, reference is made to the following descriptions taken in conjunction with the accompanying drawings, in which:

FIG. 1 shows the effect of different single stranded DNA binding proteins on delivery of Cas9 ribonucleoprotein (RNP) into EmGFP cell line (having a disrupted EmGFP gene) with a single stranded donor DNA (approximately 100 nucleotides) using electroporation.

FIG. 2A shows the effect of different single stranded DNA binding proteins on delivery of Cas9 RNP into 293FT cell line with a single stranded donor DNA (approx. 100 nucleotides) with or without nuclear localization signal (NLS) peptide attached to the donor DNA. Delivery occurred using lipid transfection (CRISPRMAX®).

FIG. 2B shows the effect of different single stranded DNA binding proteins on delivery of Cas9 mRNA into 293FT cell line with a single stranded donor DNA (approx. 100 nucleotides) with or without NLS peptide attached to the donor DNA. Delivery occurred using lipid transfection (MESSENGERMAX®).

FIG. 3 shows the effect of different modifications on delivery of Cas9 RNP into 293FT cell line with a single stranded donor DNA (approx. 1.4 kb nucleotides) using lipid transfection with CRISPRMAX®. n=control; PS=5′ phosphorothioate modification of donor DNA; TEG=5′ TEG modification of donor DNA; NLS=5′ NLS peptide conjugation; 3′ overhang=5′->3′ nuclease; SSB=single strand binding protein; Cas9-PCV=donor attached to Cas9 Porcine Circovirus HUH endonuclease fusion protein. Stars indicate P<0.05 compared to control and ssDNA alone using JPM® statistical software (SAS Institute Inc., Cary, N.C.).

FIG. 4 shows the effect of SSB on HDR efficiency in U2OS cells by flow cytometry for GFP-positive cells.

FIGS. 5A-5F show the effect of SSB on long (1.4 kb) single stranded donor DNA. U2OS cells were transfected with RNP and donor DNA using CRISPRMAX®. Each figure set shows a photomicrograph of U2OS cells under light microscopy (top panel) and fluorescent microscopy for GFP (lower panel). FIGS. 5A-5B: Double-stranded donor DNA with 5′ phosphorothioate modification. FIGS. 5C-5D: Single-stranded donor DNA. FIGS. 5E-5F: Single-stranded donor DNA with SSB.

FIG. 6 shows the effect of SSB on long (1.4 kb) single stranded donor DNA in U2OS cells, with or without C17 (NHEJ inhibitor). Cells were transfected with RNP and donor DNA using NEON™ electroporation.

FIG. 7 shows the effect of SSB on long (1.4 kb) double stranded donor DNA in U2OS cells, with or without heating. Cells were transfected with RNP and donor DNA using CRISPRMAX®. dsDNA (500 ng)/SSB (10 ug): Unmodified double-stranded donor DNA (500 ng) was mixed with 10 μg E. coli SSB without heating. dsDNA (500 ng)/SSB (10 ug)95° C.: Unmodified double-stranded donor DNA (500 ng) was mixed with 10 μg E. coli SSB, heated at 95° C. for 10 minutes, then cooled to room temperature. dsDNA (500 ng)/SSB (10 ug)95° C.4° C.: Unmodified double-stranded donor DNA (500 ng) was mixed with 10 μg E. coli SSB, heated at 95° C. for 10 minutes, then immediately put on ice. dsDNA (500 ng)/ETssb (10 ug)95° C.: Unmodified double-stranded donor DNA (500 ng) was mixed with 10 μg thermostable SSB, heated at 95° C. for 10 minutes, then cooled to room temperature.

FIG. 8 is a flowchart for ssDNA preparation using thermostable SSB.

FIG. 9 is a flowchart for ssDNA preparation using sequential exonuclease digestion.

FIG. 10A is a photograph of an agarose gel showing the kinetics of ssDNA production using the following protocol: 1.4 kb dsDNA was digested with lambda exonuclease (λExo) for 7 minutes, heated to 80° C. for 10 minutes, then digested with exonuclease III (Exo III) for 7 minutes. Gel was stained with SYBR™ Safe DNA Gel Stain.

FIG. 10B shows the fluorescence intensity of PICOGREEN® dye over time using the protocol as in FIG. 10A.

FIG. 10C shows production of ssDNA as in FIGS. 10A and 10B, based on quantitation of bottom gel bands in FIG. 10A.

FIG. 11A is a photograph of an agarose gel showing the kinetics of ssDNA production using the following protocol: 4.2 kb dsDNA was digested with lambda exonuclease (λExo) for 10 minutes, heated to 80° C. for 10 minutes, then digested with exonuclease III (Exo III) for 20 minutes. Gel was stained with SYBR™ Safe DNA Gel Stain.

FIG. 11B shows the fluorescence intensity of PICOGREEN® dye over time using the protocol as in FIG. 11A.

FIG. 11C shows production of ssDNA as in FIGS. 11A and 11B, based on quantitation of bottom gel bands in FIG. 11A.

FIGS. 12A-12I show the kinetics of ssDNA production using the following protocol: 6 kb dsDNA was digested with lambda exonuclease (λExo) for 20 minutes, then digested with exonuclease III (Exo III) for 22 minutes, without heating. Digestion was performed in the presence of 5 mM MgCl₂ (FIGS. 12A-12C), 2.5 mM MgCl₂ (FIG. 12D-12F), or 1 mM MgCl₂ (FIGS. 12G-12I). ssDNA production was monitored by agarose gel staining with SYBR™ Safe DNA Gel Stain (top row), fluorescence intensity of PICOGREEN® dye over time (FIGS. 12B, 12E, and 12H), or quantitation of bottom gel band in agarose gel (FIGS. 12C, 12F, and 12I).

FIG. 13A is a photograph of an agarose gel showing production of ssDNA from a 5 kb dsDNA without real-time monitoring and using heat inactivation.

FIG. 13B is a photograph of an agarose gel showing production of ssDNA from a 5 kb dsDNA with real-time monitoring and without heat inactivation.

FIGS. 13C and 13D show the production of ssDNA as shown in FIG. 13B, as determined by fluorescence intensity of PICOGREEN® dye over time (FIG. 13C) or quantitation of bottom gel band in FIG. 13B (FIG. 13D).

FIG. 14A is a table of NHEJ-mediated gene editing efficiency in 293FT cells with (w/SSB) or without (w/t SSB) SSB, using various Cas9 and targets. wtCas9: wild type Cas9; eCas9: high fidelity Cas9; NG-Cas9: Cas9 containing NG PAM.

FIG. 14B is a graphical representation of the normalized NHEJ-mediated editing efficiency shown in FIG. 14A.

FIG. 15 shows that using SSB as a supplement to eCas9 improves the NHEJ-mediated editing efficiency of eCas9 to near wtCas9 levels across multiple targets.

FIG. 16 shows the 178 amino acid sequence of an E. coli single stranded binding protein (SSB) (SEQ ID NO. 1). Amino acids marked with bold, italics are predicted to be involved in the binding of ssDNA. Amino acids marked with bold, italics are predicted to be involved in multimer formation.

FIG. 17 shows an alignment of amino acids of four naturally occurring SSBs. Two of the amino acid sequences are derived from bacteria of the Citrobacter genus (SEQ ID NOs. 2 and 3, respectively, in order of appearance) and two are from E. coli (SEQ ID NOs. 4 and 1, respectively, in order of appearance). The consensus sequence shown in this figure is SEQ ID NO. 19.

FIG. 18 shows the 267 amino acid sequence of a Thermus aquaticus single stranded binding protein (SSB) (SEQ ID NO. 5). Amino acids marked with bold, italics are predicted to be involved in the binding of ssDNA. Amino acids marked with bold, italics are predicted to be involved in multimer formation.

FIG. 19 shows an alignment of amino acids of three naturally occurring SSBs from different bacteria of the Thermus genus (SEQ ID NOs. 5, 6, and 7, respectively, in order of appearance). The consensus sequence shown in this figure is SEQ ID NO. 20.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or sub-value there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “single stranded DNA binding protein” refers to a DNA binding protein that binds single-stranded DNA. In some embodiments, the single stranded DNA binding protein does not have catalytic activity. In some embodiments, the single stranded DNA binding protein is non-specific in that it does not bind to a specific DNA sequence.

The term “thermostable DNA binding protein” refers to a DNA binding protein that is stable (e.g., not inactivated) at high temperature. For example, a thermostable DNA binding protein will retain at least 90% of its single stranded DNA binding activity after incubation at 75° C. for 15 minutes.

The term “exonuclease” refers to an enzyme which removes (cleaves) successive nucleotides from the end of a polynucleotide molecule. An exonuclease may cleave nucleotides from the 5′ end of the polynucleotide (“5′ to 3′ exonuclease”), or the 3′ end (“3′ to 5′ exonuclease”). Various exonucleases have different activities, for example an exonuclease may cleave only double-stranded polynucleotide, only single-stranded polynucleotides, only DNA, only RNA, both DNA and RNA, and/or may require a 3′ or 5′ modification of the polynucleotide (e.g., phosphate). Example 5′ to 3′ exonucleases include, without limitation, exonuclease II, exonuclease IV, exonuclease VIII, lambda exonuclease, RNase H, RecJ exonucleases, T7 exonuclease, Terminator 5′-Phosphate-Dependent Exonuclease, lambda exonuclease, and hSNM1 exonuclease. Example 3′ to 5′ exonucleases include, without limitation, exonuclease I, exonuclease III, exonuclease V, and Exonuclease T.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein the term “nucleic acid molecule” refers to a covalently linked sequence of nucleotides or bases (e.g., ribonucleotides for RNA and deoxyribonucleotides for DNA but also include DNA/RNA hybrids where the DNA is in separate strands or in the same strands) in which the 3′ position of the pentose of one nucleotide is joined by a phosphodiester linkage to the 5′ position of the pentose of the next nucleotide. A nucleic acid molecule may be single- or double-stranded or partially double-stranded. A nucleic acid molecule may appear in linear or circularized form in a supercoiled or relaxed formation with blunt or sticky ends and may contain “nicks”. Nucleic acid molecules may be composed of completely complementary single strands or of partially complementary single strands forming at least one mismatch of bases. Nucleic acid molecules may further comprise two self-complementary sequences that may form a double-stranded stem region, optionally separated at one end by a loop sequence. The two regions of nucleic acid molecules which comprise the double-stranded stem region are substantially complementary to each other, resulting in self-hybridization. However, the stem can include one or more mismatches, insertions or deletions. As described above, nucleic acid molecules may include chemically, enzymatically, or metabolically modified forms of nucleic acid molecules or combinations thereof. Chemically synthesized nucleic acid molecules may refer to nucleic acids typically less than or equal to 150 nucleotides long (e.g., between 5 and 150, between 10 and 100, between 15 and 50 nucleotides in length) whereas enzymatically synthesized nucleic acid molecules may encompass smaller as well as larger nucleic acid molecules as described elsewhere in the application. Enzymatic synthesis of nucleic acid molecules may include stepwise processes using enzymes such as polymerases, ligases, exonucleases, endonucleases or the like or a combination thereof.

As used herein, the term “conjugate” refers to the association between atoms or molecules. The association can be direct or indirect. For example, a conjugate between a first moiety (e.g., nuclease domain) and a second moiety (DNA binding domain) provided herein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first moiety (e.g., nuclease moiety) is non-covalently attached to the second moiety (DNA binding moiety) through a non-covalent chemical reaction between a component of the first moiety (e.g., nuclease moiety) and a component of the second moiety (DNA binding moiety). In other embodiments, the first moiety (e.g., nuclease moiety) includes one or more reactive moieties, e.g., a covalent reactive moiety, as described herein (e.g., alkyne, azide, maleimide or thiol reactive moiety). In other embodiments, the first moiety (e.g., nuclease moiety) includes a linker with one or more reactive moieties, e.g., a covalent reactive moiety, as described herein (e.g., alkyne, azide, maleimide or thiol reactive moiety). In other embodiments, the second moiety (DNA binding moiety) includes one or more reactive moieties, e.g., a covalent reactive moiety, as described herein (e.g., alkyne, azide, maleimide or thiol reactive moiety). In other embodiments, the second moiety (DNA binding moiety) includes a linker with one or more reactive moieties, e.g., a covalent reactive moiety, as described herein (e.g., alkyne, azide, maleimide or thiol reactive moiety).

The terms “genome editing” or “gene editing” as provided herein refer to stepwise processes involving enzymes such as polymerases, ligases, exonucleases, endonucleases or the like or combinations thereof. For example, gene editing may include processes where a nucleic acid molecule is cleaved, nucleotides at the cleavage site or in close vicinity thereto are excised, new nucleotides are newly synthesized and the cleaved strands are ligated.

The term “nuclear localization signal” or “NLS” refers to an amino acid sequence that ‘tags’ a protein for import into the cell nucleus by nuclear transport.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

“Contacting” is used in accordance with its plain ordinary meaning and refers to the process of allowing at least two distinct species to become sufficiently proximal to react, interact or physically touch. It should be appreciated, however, that the resulting reaction product can be produced directly from a reaction between the added reagents or from an intermediate from one or more of the added reagents which can be produced in the reaction mixture. In embodiments contacting includes, for example, allowing a nucleic acid as described herein to interact with a DNA binding protein.

A “control” sample or value refers to a sample that serves as a reference, usually a known reference, for comparison to a test sample. For example, a test sample can be taken from a test condition, e.g., in the presence of a test compound (e.g., a DNA-binding protein), and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells.

The term “gene” means the segment of DNA involved in producing a protein; it includes regions preceding and following the coding region (enhancer, promoter, leader and trailer) as well as intervening sequences (introns) between individual coding segments (exons). The enhancer, promoter, leader, trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al., Gene Therapy 8:1-4 (2001) and Prochiantz, Nat. Methods 4:119-120 (2007).

A “guide RNA” or “gRNA” as provided herein refers to a ribonucleotide sequence capable of binding a nucleoprotein, thereby forming ribonucleoprotein complex. Likewise a “guide DNA” or “gDNA” as provided herein refers to a deoxyribonucleotide sequence capable of binding a nucleoprotein, thereby forming deoxyribonucleoprotein complex. In embodiments, the guide RNA includes one or more RNA molecules. In embodiments, the guide DNA includes one or more DNA molecules. In embodiments, the gRNA includes a nucleotide sequence complementary to a target site (e.g., a modulator binding sequence). In embodiments, the gDNA includes a nucleotide sequence complementary to a target site (e.g., a modulator binding sequence). The complementary nucleotide sequence may mediate binding of the ribonucleoprotein complex or the deoxyribonucleoprotein complex to said target site thereby providing the sequence specificity of the ribonucleoprotein complex or the deoxyribonucleoprotein complex. Thus, in embodiments, the guide RNA or the guide DNA is complementary to a target nucleic acid (e.g., a modulator binding sequence). In embodiments, the guide RNA binds a target nucleic acid sequence (e.g., a modulator binding sequence). In embodiments, the guide DNA binds a target nucleic acid sequence (e.g., a modulator binding sequence). In embodiments, the guide RNA is complementary to a CRISPR nucleic acid sequence. In embodiments, the complement of the guide RNA or guide DNA has a sequence identity of about 50%, 55%, 60%, 65%, 70%, 75%, 80%, 85%, 90%, 95%, 96%, 97%, 98% or 99% to a target nucleic acid (e.g., a modulator binding sequence). A target nucleic acid sequence as provided herein is a nucleic acid sequence expressed by a cell. In embodiments, the target nucleic acid sequence is an exogenous nucleic acid sequence. In embodiments, the target nucleic acid sequence is an endogenous nucleic acid sequence. In embodiments, the target nucleic acid sequence (e.g., a modulator binding sequence) forms part of a cellular gene. Thus, in embodiments, the guide RNA or guide DNA is complementary to a cellular gene or fragment thereof. In embodiments, the guide RNA or guide DNA is about 50%, 55%, 60%, 65%, 70%, 75%, 80%, 85%, 90%, 95%, 96%, 97%, 98% or 99% to the target nucleic acid sequence (e.g., a modulator binding sequence). In embodiments, the guide RNA or guide DNA is about 50%, 55%, 60%, 65%, 70%, 75%, 80%, 85%, 90%, 95%, 96%, 97%, 98% or 99% complementary to the sequence of a cellular gene. In embodiments, the guide RNA or the guide DNA binds a cellular gene sequence. The term “target nucleic acid sequence” refers to a modulator binding sequence as provided herein.

In embodiments, the guide RNA or guide DNA is a single-stranded ribonucleic acid. In embodiments, the guide RNA or guide DNA is about 10, 20, 30, 40, 50, 60, 70, 80, 90, 100 or more nucleic acid residues in length. In embodiments, the guide RNA or guide DNA is from about 10 to about 30 nucleic acid residues in length. In embodiments, the guide RNA or guide DNA is about 20 nucleic acid residues in length. In embodiments, the length of the guide RNA or the guide DNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleic acid residues or sugar residues in length. In embodiments, the guide RNA or guide DNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more residues in length. In embodiments, the guide RNA or guide DNA is from 10 to 15, 10 to 20, 10 to 30, 10 to 40, or 10 to 50 residues in length.

For specific proteins described herein (e.g., Cas9, Argonaute), the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Thus, a “CRISPR associated protein 9,” “Cas9” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 binds a CRISPR (clustered regularly interspaced short palindromic repeats) nucleic acid sequence. In embodiments, the CRISPR nucleic acid sequence is a prokaryotic nucleic acid sequence. Examples of Cas9 proteins useful herein include without limitation, cas9 mutant proteins such as, HiFi Cas9 as described by Kleinstiver, Benjamin P., et al. (“High-fidelity CRISPR-Cas9 nucleases with no detectable genome-wide off-target effects.” Nature (2016). PubMed PMID: 26735016); Cas9 proteins binding modified PAMs and orthologous Cas9 proteins such as CRISPR from Prevotella and Francisella 1 (Cpf1). Any of the mutant Cas9 forms commonly known and described in the art may be used for the methods and compositions provided herein. Non-limiting examples of mutant Cas9 proteins contemplated for the methods and compositions provided herein are described in Slaymaker, Ian M., et al. (“Rationally engineered Cas9 nucleases with improved specificity.” Science (2015): aad5227. PubMed PMID: 26628643) and Kleinstiver, Benjamin P., et al. (“High-fidelity CRISPR-Cas9 nucleases with no detectable genome-wide off-target effects.” Nature (2016). PubMed PMID: 26735016).

As used herein “TAL effector” or “TAL effector protein” as provided herein refers to a protein including more than one TAL repeat and capable of binding to nucleic acid in a sequence specific manner. In embodiments, TAL effector protein Includes at least six (e.g., at least 8, at least 10, at least 12, at least 15, at least 17, from about 6 to about 25, from about 6 to about 35, from about 8 to about 25, from about 10 to about 25, from about 12 to about 25, from about 8 to about 22, from about 10 to about 22, from about 12 to about 22, from about 6 to about 20, from about 8 to about 20, from about 10 to about 22, from about 12 to about 20, from about 6 to about 18, from about 10 to about 18, from about 12 to about 18, etc.) TAL repeats. In embodiments, the TAL effector protein includes 18 or 24 or 17.5 or 23.5 TAL nucleic acid binding cassettes. In embodiments, the TAL effector protein includes 15.5, 16.5, 18.5, 19.5, 20.5, 21.5, 22.5 or 24.5 TAL nucleic acid binding cassettes. A TAL effector protein includes at least one polypeptide region which flanks the region containing the TAL repeats. In embodiments, flanking regions are present at the amino and/or the carboxyl termini of the TAL repeats. As used herein, the term “TALEN” refers to a TAL effector protein associated with a nuclease domain.

As used herein the term “homologous recombination” or “homology-directed repair” (or “HDR”) refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbial. Mal. Biol. Rev. 63:349-404 (1999)). In aspects, homologous recombination is enabled by the presence of said first and said second flanking element being placed upstream (5′) and downstream (3′), respectively, of said donor DNA sequence each of which being homologous to a continuous DNA sequence within said target sequence. As used herein the term “HDR-mediated genome editing” refers to genome editing that occurs through a HDR mechanism.

As used herein the term “non-homologous end joining” (NHEJ) refers to cellular processes that join the two ends of double-strand breaks (DSBs) through a process largely independent of homology. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. NHEJ (non-homologous end-joining) pathways join the two ends of a DSB through a process largely independent of homology. Depending on the specific sequences and chemical modifications generated at the DSB, NHEJ may be precise or mutagenic (Lieber M. R., The mechanism of double-strand DNA break repair by the nonhomologous DNA end-joining pathway. Annu. Rev. Biochem. 79:181-211). As used herein the term “NHEJ-mediated genome editing” refers to genome editing that occurs through a NHEJ mechanism.

As used herein the term “donor DNA” or “donor nucleic acid” refers to nucleic acid that is designed to be introduced into a locus by homologous recombination. Donor nucleic acid will have at least one region of sequence homology to the locus. In embodiments, donor nucleic acid will have two regions of sequence homology to the locus. In some embodiments, a region of sequence homology refers to a sequence that is substantially identical (more than 80%, more than 85%, more than 90%, more than 95%, more than 96%, more than 97%, more than 98%, more than 99%, or 100% identical) to the sequence of a predetermined locus in the genome of a cell. In some embodiments, each region of sequence homology is at least four (e.g., from about four to about one hundred, from about eight to about one hundred, from about ten to about one hundred, from about fifteen to about one hundred, from about twenty to about one hundred, from about thirty to about one hundred, from about four to about sixty, from about four to about one hundred, from about four to about fifty, from about four to about forty, from about four to about thirty, from about four to about twenty, from about eight to about forty, from about eight to about thirty, from about eight to about twenty, from about ten to about fifty, from about ten to about forty, from about ten to about thirty-five, from about ten to about twenty-five, etc.) nucleotides in length. These regions of homology may be at one of both termini or may be internal to the donor nucleic acid. In embodiments, an “insert” region with nucleic acid that one desires to be introduced into a nucleic acid molecule present in a cell will be located between two regions of sequence homology. In some embodiments, the insert region may be from about 1 to about 30,000 (e.g., from about 1 to about 25,000, from about 1 to about 20,000, from about 1 to about 15,000, from about 1 to about 10,000, from about 1 to about 5,000, from about 1 to about 2,000, from about 1 to about 1,000, from about 1 to about 500, from about 1 to about 200, from about 1 to about 100, from about 1 to about 75, from about 1 to about 50, from about 1 to about 25, from about 1 to about 10, etc.) nucleotides in length.

In some instances, the insert region may be relatively short. For example, one application of short insert regions is in SNP introduction or “correction. Thus, in many instances, the insert regions may be between 1 and 15 (e.g., from about 1 to about 12, from about 1 to about 8, from about 1 to about 5, from about 1 to about 3, from about 1 to about 2, from about 2 to about 12, from about 2 to about 10, from about 2 to about 8, from about 2 to about 5, from about 2 to about 3, etc.) nucleotides in length.

Donor nucleic acid molecules (e.g., donor DNA molecules) may be double-stranded, single-stranded, or partially double-stranded and single-stranded and, thus, may have overhanging termini on one or both ends (e.g., two 5′ overhangs, two 3′ overhangs, a 5′ and a 3′ overhang, a single 3′ overhang, or a single 5′ overhang). Further, nucleic acid molecules may be linear nucleic acid molecules of circular nuclei acid molecules (closed circular or nicked nucleic acid molecules.

As used herein the term “homologous recombination system or “HR system” refers components of systems set out herein that may be used to alter cells by homologous recombination. In particular, zinc finger nucleases, TAL effector nucleases, CRISPR endonucleases, homing endonucleases, and Argonaute editing systems.

As used herein, “nucleic acid cutting entity” refers to one or more molecules, enzymes, or complex of molecules with nucleic acid cutting activity (e.g., double-stranded nucleic acid cutting activity). In most embodiments, nucleic acid cutting entity components will be either proteins or nucleic acids or a combination of the two but they may be associated with cofactors and/or other molecules. The nucleic acid cutting entity will typically be selected based upon a number of factors, such as efficiency of DS break generation at target loci, the ability to generate DS break generation at suitable locations at or near target loci, low potential for DS break generation at undesired loci, low toxicity, and cost issues. A number of these factors will vary with the cell employed and target loci. A number of nucleic acid cutting entities are known in the art. For example, in some embodiments the nucleic acid cutting entity includes one or more zinc finger proteins, transcription activator-like effectors (TALEs) or transcription activator-like effector nucleases (TALENs), CRISPR complex (e.g., Cas9 or CPF1), homing endonucleases or meganucleases, argonaute-nucleic acid complexes, or macronucleases. In some embodiments, the nucleic acid cutting entity will have an activity that allows them to be nuclear localized (e.g., will contain nuclear localization signals (NLS)). In some embodiments, a single strand DNA donor could work with a nick or combination of nicks.

### Zinc Finger Proteins (ZFPs)

As used herein, “zinc finger protein” (ZFP) refers to a chimeric protein comprising a nuclease domain and a nucleic acid (e.g., DNA) binding domain that is stabilized by zinc. The individual DNA binding domains are typically referred to as “fingers,” such that a zinc finger protein or polypeptide has at least one finger, more typically two fingers, or three fingers, or even four or five fingers, to at least six or more fingers. In some embodiments, ZFPs will contain three or four zinc fingers. Each finger typically binds from two to four base pairs of DNA. Each finger may comprise about 30 amino acids zinc-chelating, DNA-binding region (see, e.g., U.S. Pat. Publ. No. 2012/0329067 A1, the disclosure of which is incorporated herein by reference).

One example of a nuclease domain is the non-specific cleavage domain from the type IIs restriction endonuclease FokI (Kim, Y. G., et al., Proc. Natl. Acad. Sci. 93:1156-60 (1996)) typically separated by a linker sequence of 5-7 base pairs. A pair of the FokI cleavage domain is generally required to allow for dimerization of the domain and cleavage of a non-palindromic target sequence from opposite strands. The DNA-binding domains of individual Cys₂His₂ ZFNs typically contain between 3 and 6 individual zinc-finger repeats and can each recognize between 9 and 18 base pairs.

One problem associated with ZFPs is the possibility of off-target cleavage which may lead to random integration of donor DNA or result in chromosomal rearrangements or even cell death which still raises concern about applicability in higher organisms (Radecke, S., et al., Mol. Ther. 18:743-753 (2010)).

### Transcription Activator-Like Effectors (TALEs)

As used herein, “transcription activator-like effectors” (TALEs) refer to proteins composed of more than one TAL repeat and is capable of binding to nucleic acid in a sequence specific manner. TALEs represent a class of DNA binding proteins secreted by plant-pathogenic bacteria of the species, such as Xanthomonas and Ralstonia, via their type III secretion system upon infection of plant cells. Natural TALEs specifically have been shown to bind to plant promoter sequences thereby modulating gene expression and activating effector-specific host genes to facilitate bacterial propagation (Römer, P., et al., Science 318:645-648 (2007); Boch, J., et al., Annu. Rev. Phytopathol. 48:419-436 (2010); Kay, S., et al., Science 3/8:648-651 (2007); Kay, S., et al., Curr. Opin. Microbiol. 12:37-43 (2009)).

Natural TALEs are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TALEs is mediated by hypervariable residues typically at positions 12 and 13 of each repeat—the so-called repeat variable diresidue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TALEs has been identified, allowing prediction of the sequential repeat order required to bind to a given DNA sequence (Boch, J., et al., Science 326:1509-1512 (2009); Moscou, M. J., et al., Science 326:1501 (2009)). Further, TAL effectors generated with new repeat combinations have been shown to bind to target sequences predicted by this code. It has been shown that the target DNA sequence generally start with a 5′ thymine base to be recognized by the TAL protein.

The modular structure of TALs allows for combination of the DNA binding domain with effector molecules such as nucleases. In particular, TALE nucleases allow for the development of new genome engineering tools.

TALEs used in some embodiments may generate DS breaks or may have a combined action for the generation of DS breaks. For example, TAL-FokI nuclease fusions can be designed to bind at or near a target locus and form double-stranded nucleic acid cutting activity by the association of two FokI domains.

In some embodiments, TALEs will contain greater than or equal to 6 (e.g., greater than or equal to 8, 10, 12, 15, or 17, or from 6 to 25, 6 to 35, 8 to 25, 10 to 25, 12 to 25, 8 to 22, 10 to 22, 12 to 22, 6 to 20, 8 to 20, 10 to 22, 12 to 20, 6 to 18, 10 to 18, 12 to 18, etc.) TAL repeats. In some embodiments, a TALE may contain 18 or 24 or 17.5 or 23.5 TAL nucleic acid binding cassettes. In additional embodiments, a TALE may contain 15.5, 16.5, 18.5, 19.5, 20.5, 21.5, 22.5 or 24.5 TAL nucleic acid binding cassettes. TALEs will generally have at least one polypeptide region which flanks the region containing the TAL repeats. In many embodiments, flanking regions will be present at both the amino and carboxyl termini of the TAL repeats. Exemplary TALEs are set out in U.S. Pat. Publ. No. 2013/0274129 A1, the disclosure of which is incorporated herein by reference, and may be modified forms on naturally occurring proteins found in bacteria of the genera Burkholderia, Xanthomonas and Ralstonia.

In some embodiments, TALE proteins will contain nuclear localization signals (NLS) that allow them to be transported to the nucleus.

### CRISPR Based Systems

The term “CRISPR” or “Clustered Regularly Interspaced Short Palindromic Repeats” is a general term that applies to three types of systems, and system sub-types. In general, the term CRISPR refers to the repetitive regions that encode CRISPR system components (e.g., encoded crRNAs). Three types of CRISPR systems (see Table 1) have been identified, each with differing features.

TABLE 1
CRISPR System Types Overview

| System | Features | Examples |
|---|---|---|
| Type 1 | Multiple proteins (5-7 proteins typical), | Staphylococcus epidermidis (Type IA) |
| | crRNA, requires PAM. DNA | |
| | Cleavage is catalyzed by Cas3. | |
| Type II | 3-4 proteins (one protein (Cas9) has | Streptococcus pyogenes CRISPR/Cas9, |
| | nuclease activity) two RNAs, requires | Francisella novicida U112 Cpf1 |
| | PAMs. Target DNA cleavage | |
| | catalyzed by Cas9 and RNA | |
| | components. | |
| Type III | Five or six proteins required for | S. epidermidis (Type IIIA); |
| | cutting, number of required RNAs | P. furiosus (Type IIIB). |
| | unknown but expected to be 1, PAMs | |
| | not required. Type IIIB systems have | |
| | the ability to target RNA. | |

As used herein, “CRISPR complex” refers to the CRISPR proteins and nucleic acid (e.g., RNA) that associate with each other to form an aggregate that has functional activity. An example of a CRISPR complex is a wild-type Cas9 (sometimes referred to as Csn1) protein that is bound to a guide RNA specific for a target locus.

As used herein, “CRISPR protein” refers to a protein comprising a nucleic acid (e.g., RNA) binding domain nucleic acid and an effector domain (e.g., Cas9, such as Streptococcus pyogenes Cas9, or CPF1 (cleavage and polyadenylation factor 1)). The nucleic acid binding domains interact with a first nucleic acid molecules either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also refers to proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein, such as Cas9 or CPF1.

In some embodiments, CRISPR proteins will contain nuclear localization signals (NLS) that allow them to be transported to the nucleus.

CRISPRs used in some embodiments may generate DS breaks or may have a combined action for the generation of DS breaks. For example, mutations may be introduced into CRISPR components that prevent CRISPR complexes from making DS breaks but still allow for these complexes to nick DNA. Mutations have been identified in Cas9 proteins that allow for the preparation of Cas9 proteins that nick DNA rather than making double-stranded cuts. Thus, some embodiments include the use of Cas9 proteins that have mutations in RuvC and/or HNH domains that limit the nuclease activity of this protein to nicking activity.

As used herein, the term “double-stranded break site” refers to a location in a nucleic acid molecule where a double-stranded break occurs. In embodiments, this will be generated by the nicking of the nucleic acid molecule at two close locations (e.g., within from about 3 to about 50 base pairs, from about 5 to about 50 base pairs, from about 10 to about 50 base pairs, from about 15 to about 50 base pairs, from about 20 to about 50 base pairs, from about 3 to about 40 base pairs, from about 5 to about 40 base pairs, from about 10 to about 40 base pairs, from about 15 to about 40 base pairs, from about 20 to about 40 base pairs, etc.). Typically, nicks may be further apart in nucleic acid regions that contain higher AT content, as compared to nucleic acid regions that contain higher GC content.

As used herein, the term “matched termini” refers to termini of nucleic acid molecules that share sequence identity of greater than 90%. A matched terminus of a DS break at a target locus may be double-stranded or single-stranded. A matched terminus of a donor nucleic acid molecule will generally be single-stranded.

As used herein, “homology directed repair” or “HDR” is a mechanism in cells to repair double-stranded breaks (DSBs) in DNA. In some embodiments, the HDR is greater than or equal to 10%, 25%, 50%, 75%, 90%, 95%, 98%, 99%, or 100%.

A common form of HDR is “homologous recombination,” which refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular, for the repair of double stranded breaks. The mechanism of homologous recombination is well known to the skilled person and has been described, for example by Paques F., Haber J. E., Microbiol. Mol. Biol. Rev. 63:349-404 (1999). In some embodiments, homologous recombination is enabled by the presence of matched termini being placed upstream (5′) and downstream (3′), respectively, in a donor nucleic acid molecule, each of which are homologous to a continuous DNA sequence within the cleaved nucleic acid molecule.

Some embodiments include compositions and methods designed to result in high efficiency of genome editing in cells (e.g., eukaryotic cells such as plant cells and animal cells, such as insect cells and mammalian cells, including mouse, rat, hamster, rabbit and human cells). In some embodiments, genome editing efficiency is such that greater than 20% of cells in a population will have underdone editing (e.g., knock-out or knock-in, such as homologous recombination) at the desired target locus or loci. In some embodiments, editing (e.g., knock-out or knock-in, such homologous recombination or other) may occur within from 10% to 65%, 15% to 65%, 20% to 65%, 30% to 65%, 35% to 65%, 10% to 55%, 20% to 55%, 30% to 55%, 35% to 55%, 40% 55%, 10% 45%, 20% to 45%, 30% to 45%, 40% to 45%, 30% to 50%, etc., of cells in a population.

Some embodiments include compositions and methods designed to result in high efficiency of homologous recombination in cells (e.g., eukaryotic cells such as plant cells and animal cells, such as insect cells and mammalian cells, including mouse, rat, hamster, rabbit and human cells). In some embodiments, homologous recombination efficiency is such that greater than 20% of cells in a population will have underdone homologous recombination at the desired target locus or loci. In some embodiments, homologous recombination may occur within from 10% to 65%, 15% to 65%, 20% to 65%, 30% to 65%, 35% to 65%, 10% to 55%, 20% to 55%, 30% to 55%, 35% to 55%, 40% 55%, 10% 45%, 20% to 45%, 30% to 45%, 40% to 45%, 30% to 50%, etc., of cells in a population.

Further, some embodiments include compositions and methods for increasing the efficiency of homologous recombination within cells. For example, if homologous recombination occurs in 10% of a cell population under one set of conditions and in 40% of a cell population under another set of conditions, then the efficiency of homologous recombination has increased by 300%. In some embodiments, the efficiency of homologous recombination may increase by 100% to 500% (e.g., 100% to 450%, 100% to 400%, 100% to 350%, 100% to 300%, 200% to 500%, 200% to 400%, 250% to 500%, 250% to 400%, 250% to 350%, 300% to 500%, etc.).

As used herein, “integration efficiency” refers to the frequency with which a segment of foreign DNA of interest is incorporated into an initial nucleic acid molecule. In some embodiments, integration efficiency of the donor nucleic acid molecule is greater than or equal to 50%, 75%, 90%, 95%, 98%, 99%, or 100%.

### Single Stranded Binding Proteins

One example of an SSB is E. coli SSB binds with high affinity in a cooperative manner to single-stranded DNA (ssDNA). After binding ssDNA, SSB is believed to destabilize helical duplexes, thereby allowing DNA polymerases to access their substrate more easily. SSB is also believed to be involved in DNA replication and recombination in vivo. SSB has also been shown to protect ssDNA from nuclease digestion.

E. coli SSBs can form homomeric multimers (e.g., multimers of four proteins subunits) and each monomer is believed to bind to eight to sixteen nucleotides. Further, the number nucleotides bound by E. coli SSBs is believed to vary with a number of factors, including salt concentration. As an example, at high salt concentration, it is believed that SSB tetramers associate with approximately 65 nucleotides of DNA but at lower salt concentrations, SSB dimers associate with approximately 35 nucleotides of DNA.

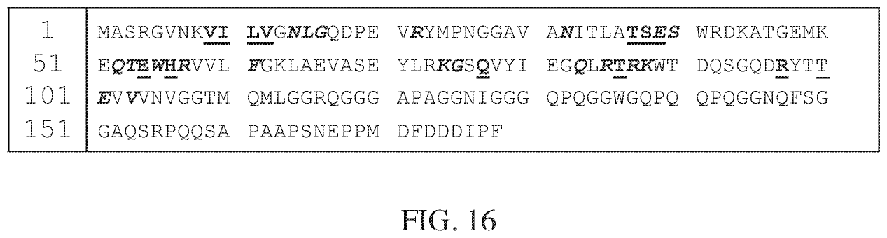

The amino acid sequence of an E. coli single stranded binding protein (SSB) is set out in FIG. 16. In some instances, SSBs used in methods and present in compositions set out herein will comprise one or more of the following amino acid regions set out in FIG. 16: 4 to 103, 9 to 103, 1 to 8, 9 to 16, 31 to 40, 52 to 61, 74 to 78, 83 to 90, and/or 97 to 103. By way of example, SSBs may comprise amino acid 9 to 16 and 52 to 61 set out in FIG. 16. Further, an SSB comprising amino acids 9 to 103 set out in FIG. 16 would also contain amino acids 31 to 40 of FIG. 16.

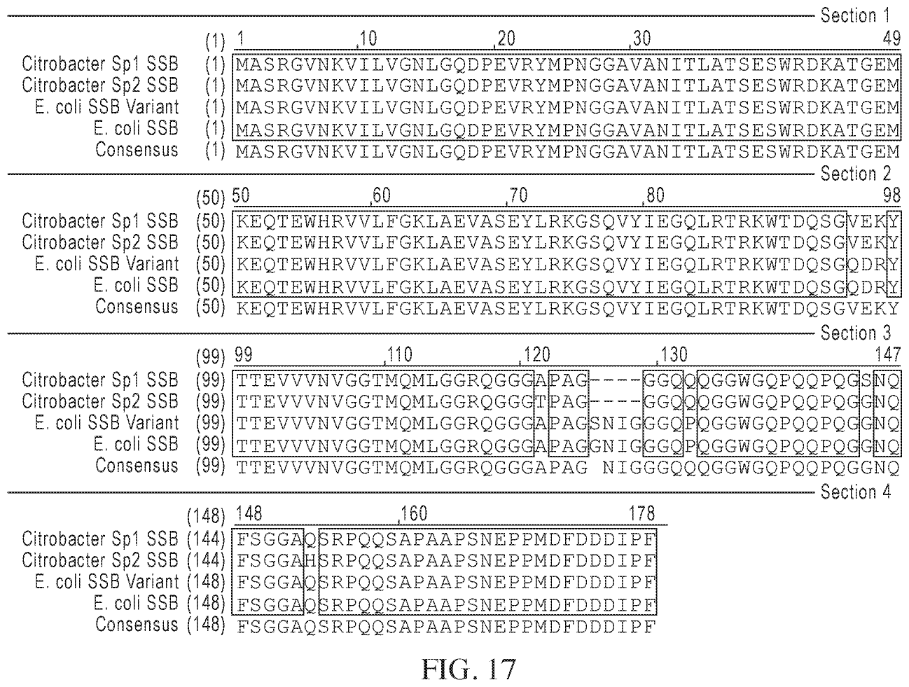

SSBs with fairly high levels of sequence homology to E. coli SSB can be found in a number of bacteria from different genera and are also encoded by mitochondrial genomes. As shown in FIG. 17, amino acid and region conservation are seen between E. coli SSB and two Citrobacter SSBs. SSBs, as well as subportions thereof, of organisms present in organisms other than E. coli may also be used in methods and present in compositions set out herein.

In some methods provided herein, thermostable SSBs may be employed (see FIG. 8 and description related thereto). Thus, compositions set out herein may also comprise thermostable SSBs.

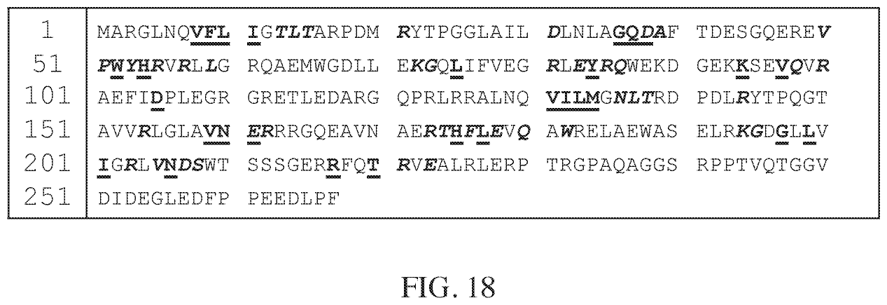

As used herein, the term “thermostable SSB” refer to an SSB that retains at least 90% of its single stranded binding activity when incubated at 75° C. for 15 minutes. One example of a thermostable SSB is a Thermus aquaticus SSB having the amino acid sequence set out in FIG. 18.

The amino acid sequence of a Thermus aquaticus single stranded binding protein (SSB) is set out in FIG. 18. In some instances, SSBs used in methods and present in compositions set out herein will comprise one or more of the following amino acid regions set out in FIG. 18: 4 to 223, 8 to 223, 1 to 6, 9 to 14, 31 to 40, 49 to 60, 71 to 76, 81 to 86, 94 to 101, 131 to 138, 154 to 162, 173 to 182, 194 to 208, and/or 217 to 223.

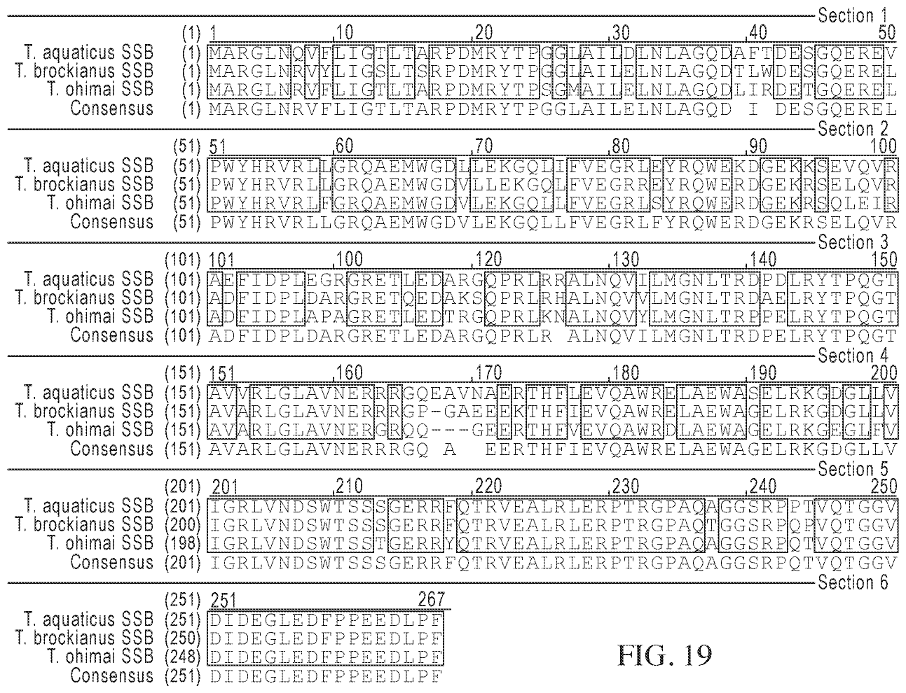

As shown in FIG. 19, amino acid and region conservation are seen between all three Thermus SSBs. Thermostable SSBs, as well as subportions thereof, of organisms present in organisms of other Thermus species as well as non-Thermus species may also be used in methods and/or present in compositions set out herein.

When SSBs are present in compositions and/or used in methods set out herein, they may be present or used in proportion to the amount of nucleic acid present. Further, the amount of SSB present will vary with considerations such as whether it is used in conjunction with ssDNA or dsDNA.

In some instances, the amount of SSB may be sufficient for saturation level binding to nucleic acid molecules present. In additional instances, the amount of SSB may be above or below such saturation level. Thus, in some instances, the amount of SSB present will vary with the nucleic acid molecules present (e.g., the number of nucleic acid molecules present, the number of nucleotides and/or base pairs of the nucleic acid molecules, etc.). As examples, the number of SSB per 100 nucleotides (or base pairs) present may be in the range of from about 12 to about 1 (e.g., from about 10 to about 1, from about 8 to about 1, from about 12 to about 8, from about 8 to about 2, from about 6 to about 2, from about 4 to about 1, etc.). By way of specific example, if a nucleic acid is 300 nucleotides (or base pairs) in length and the ratio of SSB to 100 nucleotides (or base pairs) is 5, then 15 SSBs would be present for each nucleic acid molecule present.

E. coli SSB protein is known to have high specificity ssDNA binding activity and is also believed to be involved in DNA replication, DNA recombination, and DNA repair. E. coli SSB protein is further believed to destabilize DNA secondary structure and increase the processivity of polymerases. It is thus believed that other SSB proteins, as well as protein combinations with SSB activity, with one or more activities similar to that of E. coli SSB protein may be used in compositions and methods set out herein. Examples of such proteins include helicases (e.g., DNA helicases), polymerases (e.g., DNA polymerases), exonucleases, topoisomerases, and SSBs other than E. coli SSB (e.g., T4 gene 32 protein (T4G32), alone or in combination with one or more other proteins, such as uvsX and/or uvsY proteins).

One of the functions served by helicases is the “unpackaging” an organism's DNA. Helicases are believed to be capable of moving along a nucleic acid phosphodiester backbone, separating two annealed nucleic acid strands (i.e., DNA, RNA, or RNA-DNA hybrid) using energy derived from ATP hydrolysis. About 1% of eukaryotic genes code for helicases, with the human genome encoding about 95 non-redundant helicases (64 RNA helicases and 31 DNA helicases). These proteins are believed to be involved in a number of cellular processes, such as translation, transcription, DNA replication, recombination, and DNA repair.

Exemplary helicases that may be used in compositions and methods set out here include ATP-dependent DNA helicase Q1, ATP-dependent DNA helicase Q4, ATP-dependent DNA helicase Q5, Bloom syndrome protein, Werner syndrome protein, and Twinkle T7 gp4-like protein.

Exemplary topoisomerases that that may be used compositions and methods set out here include topoisomerase IA, topoisomerase IB, topoisomerase II (e.g., gyrases), and topoisomerase IV. Specific examples of topoisomerases include E. coli topoisomerase I (e.g., New England Biolabs, cat. no. M0301S), and Vaccinia topoisomerase IB.

Compositions and methods set out herein may contain or use T4 gene 32 protein (T4G32) in conjunction with one or more additional proteins. One such protein is the E. coli virus T4 uvsX protein. Another protein is the E. coli virus T4 uvsY protein. Thus, T4G32 may be used in conjunction with one or more additional protein, resulting in, for example, the enhancement of HDR.

The amino acid sequences of a number of proteins, as well as subportions thereof, that may be used in compositions and methods set out herein are shown in Table 3.

Preparation of ssDNA

In one aspect, a method for preparing single stranded DNA (ssDNA) is provided. ssDNA is often a better donor than dsDNA for homologous recombination (e.g., in gene editing reactions) because it is not randomly recombined into the genome. This is important for therapeutic use, where random recombination is a potential safety concern. However, donor DNA can range in size from a few hundred bases to several kilobases (kb)—too large to be made by most synthesis methods.

ssDNA is often made from dsDNA, which can be economically and quickly made by many different methods. Exonucleases (e.g., lambda exonuclease) can be used to digest a first strand of the dsDNA while leaving the other, complementary strand intact. Generally, the first strand is prepared in a way that permits the exonuclease to digest the strand. For example, the first strand can be modified (e.g., by 5′ phosphate) so that it is cleaved/digested by the exonuclease, while the complimentary strand is unmodified and resistant to cleavage. However, these exonucleases, in particular lambda exonuclease, do not always completely cleave the first DNA strand. For example, the exonuclease may “stall” during cleavage and not restart, or may fall off the DNA. This results in DNA that is only partly single-stranded.

To combat this problem, a second exonuclease can be used subsequent to the first. If the first exonuclease is a 5′ to 3′ exonuclease, then the second exonuclease can be a 3′ to 5′ exonuclease (and vice versa). The second exonuclease preferably requires dsDNA for cleavage, to avoid cleaving the complementary ssDNA strand. For example, exonuclease III is a 3′ to 5′ exonuclease that only cleaves from the 3′ end of dsDNA. See, e.g., U.S. Patent Pub. 2017/0349927, which is incorporated herein by reference in its entirety, including for all reagents, methods, compositions, etc. described therein.

Surprisingly, it has been discovered that heat inactivation of the first exonuclease prior to addition of the second exonuclease results in reduced quality of the resulting ssDNA product. Also, determining when to add the second exonuclease, and also when to stop the reaction after addition of the second exonuclease, is problematic. If the reaction goes too long, the exonucleases can start cleaving the ssDNA, resulting in lower yield; stopping the reaction too soon can result in partially single-stranded DNA. The technology described herein addresses these potential issues in some instance by, for example, eliminating the heat inactivation steps, eliminating buffer changes between the two exonuclease reactions, and/or monitoring the digestion in real time.

In embodiments, the method for preparing ssDNA includes (a) providing a composition comprising a double stranded DNA (dsDNA); (b) contacting the composition with a first exonuclease and (c) contacting the composition comprising the first exonuclease with a second exonuclease. In some embodiments, the first exonuclease is a 5′ to 3′ exonuclease (e.g., lambda exonuclease). In some embodiments, the second exonuclease is a 3′ to 5′ exonuclease (e.g., exonuclease III). In embodiments, step (b) and step (c) are performed without changing buffer. In embodiments, step (c) is performed without inactivating the first exonuclease.

In embodiments, steps (b) and (c) are performed under conditions where each exonuclease will cleave/digest the intended strand of the dsDNA. In embodiments, the strand intended for digestion can be modified such that the first and/or second exonuclease will preferentially digest the intended strand, and not the complementary strand. In embodiments, the complementary strand can be modified such that the first and/or second exonuclease will not digest the complementary strand. In embodiments, the steps are performed in the presence of a buffer that is compatible with both exonucleases (e.g., both exonucleases function in the buffer).

Throughout this disclosure, where a “first exonuclease” and a “second exonuclease,” it is to be understood that a “first exonuclease” and a “second exonuclease” could be used together. Thus, in embodiments, the method for preparing ssDNA includes (a) providing a composition comprising a double stranded DNA (dsDNA); (b) contacting the composition with a first exonuclease; and (c) contacting the composition comprising the first exonuclease with a second exonuclease. In embodiments, the first exonuclease is a 3′ to 5′ exonuclease, and the second exonuclease is a 5′ to 3′ exonuclease, and step (b) and step (c) are performed without changing buffer. In embodiments, step (c) is performed without inactivating the first exonuclease.

In embodiments, the dsDNA has a modification on one strand. Such modifications may be protective, meaning the difference protects one strand from degradation, or permissive, meaning that the difference allows a particular strand to be degraded. Protective and permissive modifications are known in the art. In embodiments, the modification is a 3′ overhang, a 5′ overhang, a blunt end, a phosphate, a modified base (e.g., a phosphorothioate internucleotide linkage), reverse nucleotide linkages (e.g., a 3′-3′ linkage or a 5′-5′ linkage), 2′-O-Methyl base modifications (e.g., 2′-O Methyl Adenosine, 2′-O Methyl Cytosine, 2′-O Methyl Guanosine, 2′-O Methyl Uridine, etc.), 2′ Fluoro base modifications (e.g., 2′-Fluoro deoxyadenosine, 2′-Fluoro deoxycytosine, 2′-Fluoro deoxyguanosine, 2′-Fluoro deoxyuridine, etc.), propyne modified nucleotides (e.g., propyne dC deoxycytosine, propyne dU deoxyuridine, etc.), phosphorylation modifications, methylphosphonate modifications, multi-carbon spacer modifications (e.g., C3 spacer modifications, (e.g., C3 Spacer Amidite (DMT-1,3-Propanediol), 1-(4,4′-Dimethoxytrityloxy)-propanediol-3-succinoyl-lcaa-CPG, 3-(4,4′-Dimethoxytrityloxy)propyl-1-[(2-cyanoethyl)-(N,N-diisopropyl)]-phosphoramidite, etc.), C9 Spacer modifications (e.g., 8-O-(4,4′-Dimethoxytrityl)-triethyleneglycol, 1-[(2-cyanoethyl)-(N,N-diisopropyl)]-phosphoramidite, etc.) C12 Spacer modifications (e.g., 12-(4,4′-Dimethoxytrityloxy)dodecyl-1-[(2-cyanoethyl)-(N,N-diisopropyl)]-phosphoramidite, etc.), and the like), dephosphorylation, or labels (e.g., biotin label, fluorescent label). In embodiments, the dsDNA has a modification on the 5′ end of one strand. In embodiments, the 5′ modification is a 5′ phosphate. In embodiments, the first 5′ to 3′ exonuclease only digests the strand having the modification. In embodiments, the first exonuclease is a 5′ to 3′ exonuclease that only digests the strand without the modification. In embodiments, any one or more of the modifications are specifically excluded.

Exonucleases that can be used as described herein include 3′ to 5′ exonucleases. Examples of 3′ to 5′ exonucleases include, without limitation, exonuclease I, exonuclease III, exonuclease V, and Exonuclease T.

Exonucleases that can be used as described herein include 5′ to 3′ exonucleases. Examples of 5′ to 3′ exonucleases include, without limitation, exonuclease II, exonuclease IV, exonuclease VIII, lambda exonuclease, RNase H, RecJ exonucleases, T7 exonuclease, Terminator 5′-Phosphate-Dependent Exonuclease, lambda exonuclease, and hSNM1 exonuclease. In some embodiments, the 5′ to 3′ exonuclease is dependent upon (or prefers) the presence of a 5′ phosphate.

Additional exonucleases that can be used include, without limitation, those described in Spitzer & Eckstein (1988) Nucleic Acids Res. 16(24): 11691-11704 (incorporated herein by reference in its entirety), and the like.

In embodiments, the exonuclease is exonuclease II. In embodiments, the exonuclease is exonuclease IV. In embodiments, the exonuclease is exonuclease V. In embodiments, the exonuclease is exonuclease VIII. In embodiments, the first or second exonuclease is RNase H exonuclease. In embodiments, the first or second exonuclease is lambda exonuclease. In embodiments, the first or second exonuclease is Exonuclease III. The exonuclease may be a variant or modified version of any of the listed exonucleases. In embodiments, any one or more of the exonucleases listed herein are specifically excluded.

In embodiments, the second exonuclease preferentially cleaves only the strand that was cleaved by the first exonuclease. In embodiments, the second exonuclease is blocked by a protective modification. In embodiments, the second exonuclease only digests dsDNA (e.g., dsDNA without a 5′ overhang).

In embodiments, the composition includes a detectable marker that binds to a nucleotide. In embodiments, the detectable marker preferentially interacts with ssDNA. In embodiments, the detectable marker preferentially interacts with dsDNA. In embodiments, the detectable marker is a fluorescent nucleic acid stain. In embodiments, the fluorescent nucleic acid stain is PICOGREEN™. In embodiments, the detectable marker is QUANTIFLUOR® dsDNA dye (Promega Corporation, cat. no. E2670).

In embodiments, the method includes monitoring an amount of detectable marker. In embodiments, the amount of detectable marker is monitored in real time. In embodiments, the amount of detectable marker is monitored by QUBIT™ Fluorometric Quantification or similar system. In embodiments, the amount of detectable marker is monitored by QUANTIFLUOR® dsDNA System (Promega) or similar system.

In embodiments, the second exonuclease is added when the amount of ssDNA reaches a plateau. In embodiments, the second exonuclease is added when the amount of dsDNA reaches a plateau. In embodiments, the second exonuclease is added when the amount of the detectable marker reaches a plateau.

In embodiments, the reaction is stopped when the amount of ssDNA reaches a plateau. In embodiments, the reaction is stopped when the amount of dsDNA reaches a plateau. In embodiments, the reaction is stopped when the amount of the detectable marker reaches a plateau.

In embodiments, the temperature of the composition does not exceed about 60° C. In embodiments, the temperature of the composition does not exceed about 5° C. In embodiments, the temperature of the composition is maintained between about 20° C. and about 50° C. In embodiments, the temperature of the composition is maintained between about 30° C. and about 40° C. In embodiments, the temperature of the composition is maintained at about 37° C. In embodiments, the temperature of the composition is maintained at about room temperature. In embodiments, the temperature is maintained at the indicated temperature/range throughout the method. The temperature can be maintained at any value or within any subrange within the recited ranges, including endpoints.

In embodiments, step (b) is performed for a period of time sufficient for at least partial digestion of a strand of the dsDNA. In embodiments, step (c) is performed for a period of time sufficient for at least partial digestion of a strand of the dsDNA. The amount of time required for digestion depends on the length of the dsDNA and/or the type of exonuclease used. In embodiments, the period of time is determined by real time monitoring of the detectable marker.

In embodiments, the method includes (d) adding a stop buffer. Stop buffer stops the reaction (e.g., the cleavage by one or more of the exonucleases). In embodiments, the stop buffer includes EDTA. In embodiments, the stop buffer includes 0.1 M to 1 M EDTA. In embodiments, the stop buffer includes about 0.5 M EDTA.

In embodiments, the ssDNA is purified after step (c) or (d). Any DNA purification method can be used. Non-limiting examples include silica membrane column, silica-coated particles (e.g., paramagnetic particles), agarose gel purification, etc.

In embodiments, the dsDNA is between 100 and 10,000 base pairs in length. In embodiments, the dsDNA is between 100 and 1,000 base pairs in length. In embodiments, the dsDNA is between 100 and 2,000 base pairs in length. In embodiments, the dsDNA is between 100 and 3,000 base pairs in length. In embodiments, the dsDNA is between 100 and 4,000 base pairs in length. In embodiments, the dsDNA is between 100 and 5,000 base pairs in length. In embodiments, the dsDNA is between 100 and 6,000 base pairs in length. In embodiments, the dsDNA is between 100 and 7,000 base pairs in length. In embodiments, the dsDNA is between 100 and 8,000 base pairs in length. In embodiments, the dsDNA is between 100 and 9,000 base pairs in length. In embodiments, the dsDNA is between 500 and 10,000 base pairs in length. In embodiments, the dsDNA is between 1,000 and 10,000 base pairs in length. In embodiments, the dsDNA is between 2,000 and 10,000 base pairs in length. In embodiments, the dsDNA is between 3,000 and 10,000 base pairs in length. In embodiments, the dsDNA is between 4,000 and 10,000 base pairs in length. In embodiments, the dsDNA is between 5,000 and 10,000 base pairs in length. In embodiments, the dsDNA is between 6,000 and 10,000 base pairs in length. In embodiments, the dsDNA is between 7,000 and 10,000 base pairs in length. In embodiments, the dsDNA is between 8,000 and 10,000 base pairs in length. In embodiments, the dsDNA is between 9,000 and 10,000 base pairs in length. The length of the dsDNA can be any value or subrange within the recited ranges, including endpoints.

In embodiments, the composition includes magnesium. In embodiments, the composition includes between 1 mM and 10 mM MgCl₂. In embodiments, the composition includes between 1 mM and 9 mM MgCl₂. In embodiments, the composition includes between 1 mM and 8 mM MgCl₂. In embodiments, the composition includes between 1 mM and 7 mM MgCl₂. In embodiments, the composition includes between 1 mM and 6 mM MgCl₂. In embodiments, the composition includes between 1 mM and 5 mM MgCl₂. In embodiments, the composition includes between 1 mM and 4 mM MgCl₂. In embodiments, the composition includes between 1 mM and 3 mM MgCl₂. In embodiments, the composition includes between 1 mM and 2.5 mM MgCl₂. In embodiments, the composition includes about 1 mM MgCl₂. In embodiments, the composition includes about 2 mM MgCl₂. In embodiments, the composition includes about 2.5 mM MgCl₂. In embodiments, the composition includes about 3 mM MgCl₂. In embodiments, the composition includes about 4 mM MgCl₂. In embodiments, the composition includes about 5 mM MgCl₂. In embodiments, the composition includes about 6 mM MgCl₂. The concentration can be any value or subrange within the recited ranges, including endpoints.

In an aspect, a composition including dsDNA, a first exonuclease, e.g., a 5′ to 3′ exonuclease (e.g., lambda exonuclease), a second exonuclease, e.g., a 3′ to 5′ exonuclease (e.g., exonuclease III), and a detectable marker that binds to nucleotides is provided. In embodiments, the composition includes magnesium. In embodiments, the composition includes between 1 mM and 10 mM MgCl₂.

In an aspect, a kit including a first exonuclease (e.g., a 5′ to 3′ exonuclease, such as lambda exonuclease), a second exonuclease (e.g., a 3′ to 5′ exonuclease, such as exonuclease III), and a detectable marker that binds to nucleotides is provided. In embodiments, the kit includes a magnesium salt. In embodiments, the kit includes a reaction buffer, such that the first exonuclease and/or second exonuclease have enzymatic activity when present in the reaction buffer. In embodiments, both the first exonuclease and the second exonuclease have enzymatic activity when present in the reaction buffer. In embodiments, the reaction buffer contains one or more of Tris, MgCl₂, DMSO, Triton X-100, and PicoGreen. In embodiments, the reaction buffer contains one or more of 1 mM to 100 mM TrisHCl (pH 7-9), 0.5 mM to 50 mM MgCl₂, 1% to 20% (v/v) DMSO, 0.005% to 0.1% (v/v) Triton X-100, and 1:5,000 to 1:50,000 dilution of PICOGREEN®. In embodiments, the reaction buffer contains about 66 mM TrisHCl (pH about 8.5), about 2.5 mM, MgCl₂, about 10% DMSO, about 0.01% Triton X-100, and about 1:20,000 dilution of PICOGREEN®.

In an aspect, a system including (a) a device configured to detect a detectable marker that binds to a nucleotide; and (b) a composition comprising DNA, an exonuclease, and a detectable marker that binds to nucleotides. In embodiments, the exonuclease is a lambda exonuclease and/or exonuclease III. In embodiments, the exonuclease is a lambda exonuclease and exonuclease III.

In embodiments, the detectable marker is a fluorescent nucleic acid stain. In embodiments, the detectable marker preferentially interacts with ssDNA. In embodiments, the detectable marker preferentially interacts with dsDNA. In embodiments, the detectable marker is a fluorescent nucleic acid stain. In embodiments, the fluorescent nucleic acid stain is PICOGREEN™.

In an aspect, a method for preparing a single stranded target DNA (ssDNA) is provided. In embodiments, the method includes (a) providing a template DNA comprising a target donor DNA sequence; (b) providing an amplification primer pair comprising a forward primer and a reverse primer designed to amplify the target donor DNA sequence, wherein the forward primer comprises a 5′ end comprising ribonucleotides and a 3′ end comprising deoxynucleotides, and wherein the reverse primer is not susceptible to digestion by RNase H; (c) amplifying the target donor DNA sequence with the forward and reverse primers to generate an amplification product comprising a first strand and a second strand complementary to the first strand, wherein the 5′ end of the first strand of the amplification product is susceptible to digestion by RNaseH; (d) contacting the amplification product with an RNaseH exonuclease, and; (e) contacting the amplification product with a second exonuclease, e.g. a 5′ to 3′ exonuclease. In embodiments, steps (c) and (d) are simultaneous. In embodiments, steps (c) and (d) are sequential.

In embodiments, the method includes contacting the amplification product with a third exonuclease.

In embodiments, the second exonuclease is Lambda exonuclease. In embodiments, the third exonuclease is a 3′ to 5′ exonuclease. In embodiments, the third exonuclease is Exonuclease III.

In embodiments, the amplification product is contacted with the RNaseH, second exonuclease, and third exonuclease simultaneously.

In embodiments, the amplification product is generated by polymerase chain reaction (PCR).

In an aspect a method for preparing single stranded DNA (ssDNA) is provided. In embodiments, the method includes denaturing a double stranded DNA (dsDNA) in the presence of a single-strand DNA binding protein, thereby preparing ssDNA. In embodiments, the dsDNA is denatured by heating.

Without being bound by theory, it is believed that the single-strand DNA binding protein will bind each strand of the denatured DNA molecule, thereby preventing the individual strands from re-annealing after the denaturation step (e.g., during cooling).

In embodiments, the single-strand DNA binding protein is SSB. In embodiments, the SSB is a thermostable SSB. In embodiments, the single strand DNA binding protein comprises the E. coli SSB C-terminus or variant thereof.

In embodiments, one strand of the dsDNA is labeled. In embodiments, the method includes isolating the labeled strand. In embodiments, the method includes depleting the labeled strand. The label may be any molecule that allows preferential retention of the labeled strand, for example biotin. Where the label is biotin, the labeled strand can be isolated (or depleted) using a streptavidin column or streptavidin-coated beads.

FIG. 8. shows an exemplary set of methods for the generation of ssDNA. In these exemplary methods, dsDNA with a biotin moiety on one terminus of one strand is heat denatured in the presence of a thermostable SSB. The thermostable SSB then binds to the ssDNA molecules, preventing them from rehybridizing with each other. The left side of the figure shows the direct use of the ssDNA molecules by introduction into cells. The terminal biotin moiety may be omitted in such methods. The right side of FIG. 8 shows a process by which the two DNA strands of the dsDNA molecule are separated from each other. In this embodiment, the 5′ terminus of one strand of the dsDNA molecule is covalently linked to a biotin moiety. This allows for strand separation because one strand of the original dsDNA molecule can be sequestered by interaction with a solid support comprising streptavidin. Further, either of the strands may then be collected for downstream use (e.g., introduction into a cell). The non-biotinylated strand may be collected in the unbound fraction (e.g., supernatant, column effluent, etc.). The biotinylated strand may be collected by release from the solid support by any number of methods (e.g., biotin competition based release).

Provided herein are methods for the separation of two strands of a dsDNA molecule, as well as composition that may be used in such methods. In some instances, such methods include methods for generating a composition comprising separated of strands of a double-stranded DNA molecule, these methods comprise denaturing the dsDNA molecule in the presence of a single-stranded binding protein (e.g., a thermostable SSB), wherein the denaturing is mediated by heating the dsDNA molecule to a temperature above its melting temperature (see FIG. 8, left side).

Of course, the melting temperature of the dsDNA molecule used in such methods will vary with a number of factors, including the GC content. Further, one or both termini of one or both strands of the dsDNA molecule may comprise a purification moiety (e.g., biotin) or no purification moiety may be present.

As suggested above, in some instances, strand separation methods include the use of capture moiety (e.g., biotin). In such methods, these methods comprise denaturing the dsDNA molecule in the presence of a single-stranded binding protein (e.g., a thermostable SSB), followed by capture of one of the strands through interaction with the capture moiety (see FIG. 8, right side).

### Genetic Modification

In an aspect a method for genetically modifying a cell is provided. In some embodiments, the genetic modification comprises introduction of a double-stranded break at a target sequence in the genome. In some embodiments, a method for genetically modifying a cell to disrupt, or “knock-out” a gene is provided. In such embodiments, the double stranded break which is repaired by the cellular machinery, and wherein the repair comprises non-homologous end joining (NHEJ) that introduces errors in the target sequence in the genome. In some embodiments, the genetic modification refers to integration of a donor nucleic acid sequence (e.g., a donor DNA sequence) at a predetermined locus in the cell. In such embodiments, the method includes introduction of a double-stranded break at a target sequence in the genome, which is subsequently repaired by the cellular machinery, and wherein the repair comprises homology-directed repair (HDR) that introduces a donor sequence into the genome of the cell at the desired location (i.e., a “knock-in”). HDR-mediated genetic modification using a nucleic acid cutting entity uses a donor DNA to introduce the desired modification.

In embodiments wherein the genetic modification comprises HDR, donor DNA may be ssDNA or dsDNA. One limitation is that the donor DNA is often degraded by cells before it can be integrated by HDR. Without being bound by theory, it is believed that the present method protects the donor DNA during the process of introducing it into the cell and initiating HDR.

In an aspect a method for improving targeting efficiency of a nucleic acid cutting entity for genetic modification of a cell via HDR is provided. In an aspect a method for reducing off-target integration of a donor DNA during HDR-mediated genetic modification of a cell is provided. In an aspect a method for enhancing delivery of a donor DNA to a cell for genetic modification of the cell is provided. In an aspect a method for reducing degradation of a donor DNA for genetic modification of a cell is provided.

In embodiments, the method includes introducing into the cell: (i) at least one donor DNA molecule; (ii) at least one single stranded DNA binding protein; and (iii) at least one nucleic acid cutting entity or nucleic acid encoding the at least one nucleic acid cutting entity; under conditions that allow for genetically modifying the cell at a predetermined locus.

In an aspect, a method for genetically modifying a cell is provided. In embodiments, the method includes introducing into the cell and at least one non-specific single strand DNA binding protein or nucleic acid encoding the at least one non-specific single stranded DNA binding protein in the presence of at least one nucleic acid cutting entity or nucleic acid encoding the at least one nucleic acid cutting entity; under conditions that allow for genetically modifying the cell at a predetermined locus.

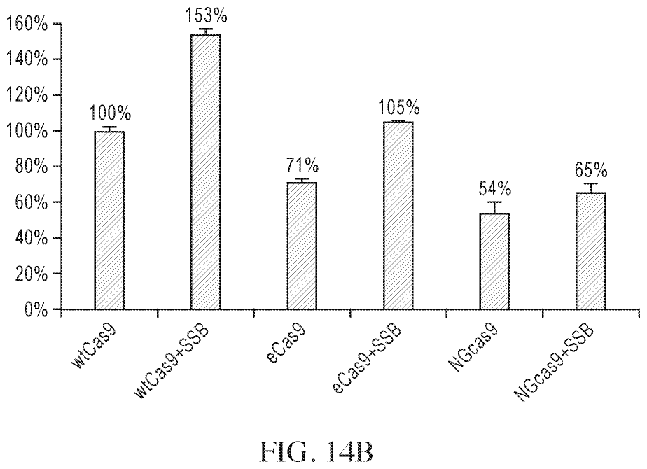

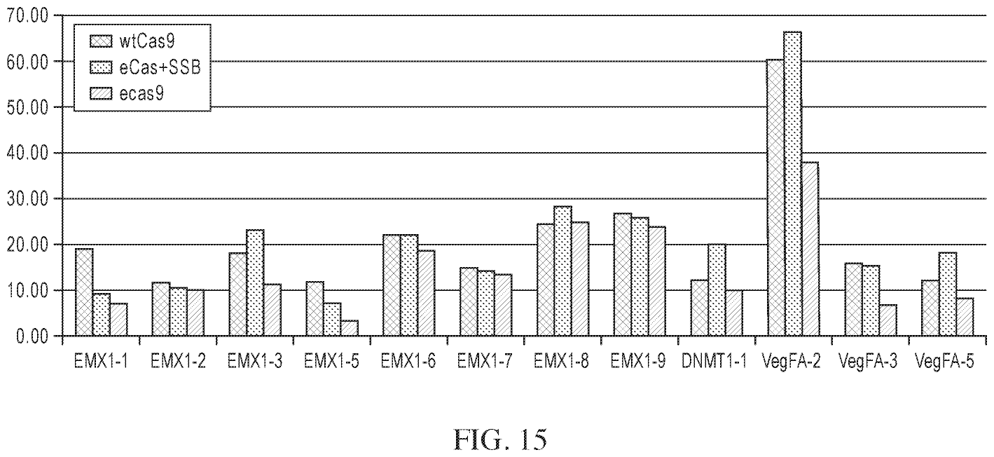

Further set out herein are compositions and methods for enhancing the efficiency of gene (e.g., genome) editing. Comparative data related to such composition and methods are set out in FIGS. 14A-14B and FIG. 15.

It has been found that association of donor DNA with SSBs can yield increased gene editing efficiency. As an example, the data set out in FIG. 14A shows normalized average increases of 153% and 148% using two different Cas9 proteins. These data were generated through measurement of gene editing at 12 different target sites. Thus, 24 data points were generated with 23 data points showing increased editing.

Further provided herein are methods for enhancing gene editing efficiency (e.g., enhancing genome editing efficiency), as well as compositions used in such methods. In some aspects, these methods may comprise: (a) contacting donor DNA (e.g., single stranded donor DNA) with one or more SSB, under conditions that allow for the association of the SSB with the donor DNA to form a donor DNA-SSB complex; and (b) introducing the a donor DNA-SSB complex into a cell. In many instances, the cell will also be contacted with a nucleic acid cutting entity (e.g., a TALE, Cas9/gRNA combinations, etc.) with cleavage specificity within or near (e.g., within 10 base pairs of one terminus of the donor DNA) a genetic region with sequence homology to the donor DNA.

The amount of SSB with respect to the amount of donor DNA used in such methods and compositions is set out elsewhere herein. Further, these amounts (e.g., ratios) will often be adjusted to result in enhanced editing efficiency.

One method for determining increased editing efficiency is by performing gene editing under two set of conditions. For example, gene editing efficiency may be determined using donor associated with one or more SSB and not associated with an SSB (see FIGS. 14A-14B and FIG. 15, and description related thereto). Methods set out herein may result in editing efficiency increase of from about 10% to about 200% (e.g., from about 10% to about 180%, from about 10% to about 160%, from about 10% to about 120%, from about 10% to about 100%, from about 10% to about 80%, from about 10% to about 70%, from about 30% to about 120%, from about 40% to about 90%, etc.). Using data set out in FIG. 14A for purposes of illustration, the percent increases in editing efficiency are 53% and 48%. These number are calculated as follows: Final Value (153%)— Original Value (100%)/Original Value (100%)=Percent Increase (53%).

In some embodiments, the method provides for genetically modifying a cell via NHEJ-mediated gene editing.

In some embodiments, the method provides for genetically modifying a cell via HDR-mediated gene editing. In such embodiments, the method can further comprise introducing into the cell a donor DNA.

In embodiments, one or more of the at least one nucleic acid cutting entity is a zinc finger nuclease; a TAL effector nuclease; and/or a CRISPR complex. In embodiments, one or more of the at least one nucleic acid cutting entity is a CRISPR complex. In embodiments, the CRISPR complex is a Cas9/gRNA complex.

In embodiments, the donor DNA is associated with the DNA binding protein prior to introduction into the cell. In embodiments, the donor DNA molecule is contacted with the at least one single stranded DNA binding protein prior to introduction into the cell. In embodiments, the donor DNA and the at least one single stranded DNA binding protein are incubated together for a period of time before introducing them into a cell. In embodiments, the period of time is sufficient for interaction (e.g., binding) between the at least one single stranded DNA binding protein and the donor DNA to occur.

In embodiments, the at least one nucleic acid cutting entity or nucleic acid encoding the at least one nucleic acid cutting entity is introduced into the cell before introduction of the donor DNA and/or at least one non-specific single strand DNA binding protein or nucleic acid encoding the at least one non-specific single stranded DNA binding protein into the cell. In embodiments, the at least one nucleic acid cutting entity or nucleic acid encoding the at least one nucleic acid cutting entity is introduced into the cell after introduction of the donor DNA and/or at least one non-specific single strand DNA binding protein or nucleic acid encoding the at least one non-specific single stranded DNA binding protein into the cell.

In embodiments, the single-strand DNA binding protein comprises an oligonucleotide/oligosaccharide-binding (OB)-fold. In embodiments, the single-strand DNA binding protein is SSB, RecA, or T4G32. In embodiments, the single-strand DNA binding protein is SSB or a variant thereof. In embodiments, the SSB is E. coli SSB or variant thereof. In embodiments, one or more of the at least one single-strand DNA binding protein comprises the E. coli SSB C-terminus or variant thereof. In embodiments, one or more of the at least one single-strand DNA binding protein comprises a DNA binding domain of a single-strand DNA binding protein. In embodiments, the E. coli SSB or variant thereof includes a N-terminal domain containing multiple basic residues or positively charged amino acids, for example for DNA binding. In embodiments, the E. coli SSB or variant thereof includes a C-terminal domain containing multiple negatively charged or acidic amino acids, for example for interaction with other SSB binding proteins. In embodiments, the single-strand DNA binding protein binds to ssDNA every 30 to 73 nucleotides, depending on the salt concentration.

In embodiments, the DNA binding protein is present in an amount sufficient to protect the donor DNA from degradation. In embodiments, the DNA binding protein is present in an amount sufficient to improve the targeting efficiency of the at least one nucleic acid cutting entity. In embodiments, the molar ratio of the DNA binding protein over DNA ranges from 0.1 to 1000. In embodiments, the molar ratio of E. coli SSB over 1.5 kb ssDNA or dsDNA is between 3 to 300.

In embodiments, the donor DNA comprises a nuclear localization signal (NLS). In embodiments, the DNA binding protein comprises a nuclear localization signal (NLS). In embodiments, the NLS peptide(s) can be placed at the 5′ end of the donor DNA. In embodiments, the NLS peptide(s) can be placed at the 3′ end of the donor DNA. In embodiments, the NLS peptide(s) can be placed internal to the donor DNA.

In embodiments, the donor DNA and single-strand DNA binding protein are introduced into the cell using transfection. In embodiments, the donor DNA and single-strand DNA binding protein are introduced into the cell using lipid transfection. In embodiments, the donor DNA and single-strand DNA binding protein are introduced into the cell using electroporation. In embodiments, the donor DNA and single-stranded DNA binding protein are introduced into the cells using CaSO₄ or polyethylenimine (PEI).

In some embodiments, a reagent for the introduction of macromolecules into cells can comprise one or more lipids which can be cationic lipids and/or neutral lipids. Preferred lipids include, but are not limited to, N-[1-(2,3-dioleyloxy) propyl]-N,N,N-trimethylamonium chloride (DOTMA), dioleoylphosphatidylcholine (DOPE), 1,2-Bis(oleoyloxy)-3-(4′-trimethylammonio) propane (DOTAP), dihydroxyl-dimyristylspermine tetrahydrochloride (DHDMS), hydroxyl-dimyristylspermine tetrahydrochloride (HDMS), 1,2-dioleoyl-3-(4′-trimethylammonio) butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), 1,2-dioleoyl-3-dimethyl-hydroxyethyl ammonium bromide (DORI), 1,2-dioleyloxypropyl-3-dimethyl-hydroxyethyl ammonium bromide (DOME), 1,2-dimyristyloxypropyl-3-dimethylhydroxyethyl ammonium bromide (DMRIE), O,O′-didodecyl-N-[p(2-trimethylammonioethyloxy)benzoyl]-N,N,N-trimethylammonium chloride, spermine conjugated to one or more lipids (for example, 5-carboxyspermylglycine dioctadecylamide (DOGS), N,N^{I},N^{II},N^{III}-tetramethyl-N,N^{I},N^{II},N^{III}-tet-rapalmitylspermine (TM-TPS) and dipalmitoylphasphatidylethanolamine 5-carboxyspermylaminde (DPPES)), lipopolylysine (polylysine conjugated to DOPE), TRIS (Tris(hydroxymethyl)aminomethane, tromethamine) conjugated fatty acids (TFAs) and/or peptides such as trilysyl-alanyl-TRIS mono-, di-, and tri-palmitate, (3B-[N-(N′,N′-dimethylaminoethane)-carbamoyl] cholesterol (DCChol), N-(α-trimethylammonioacetyl)-didodecyl-D-glutamate chloride (TMAG), dimethyl dioctadecylammonium bromide (DDAB), 2,3-dioleyloxy-N-[2(spermine-carboxamido)ethyl]-N,N-dimethyl-1-propanamin-iniumtrinuoroacetate (DOSPA) and combinations thereof.

Those skilled in the art will appreciate that certain combinations of the above mentioned lipids have been shown to be particularly suited for the introduction of nucleic acids into cells for example a 3:1 (w/w) combination of DOSPA and DOPE is available from Life Technologies Corporation, Carlsbad, Calif. under the trade name LIPOFECTAMINE™, a 1:1 (w/w) combination of DOTMA and DOPE is available from Thermo Fisher Scientific under the trade name LIPOFECTIN®, a 1:1 (M/M) combination of DIVIRIE and cholesterol is available from Life Technologies Corporation, Carlsbad, Calif. under the trade name DIVIRIE-C reagent; a 1:1.5 (M/M) combination of TM-TPS and DOPE is available from Life Tech. In some embodiments, the transfection reagent is a cationic lipid transfection reagent. In some embodiments, the transfection reagent is a polymer-based transfection reagent. Other commercially available cationic lipid transfection reagents include, without limitation, TRANSFAST™ (available from Promega Corporation); LYOVEC™ (available from INVIVOGEN™); DOTAP liposomal transfection reagent (available from Roche); TRANSIT® transfection reagents (available from Mirius Bio); and Insect GENEJUICE® Transfection Reagent (EMD Millipore). Additional transfection reagents that may be used herein include, without limitation, LIPOFECTAMINE™, LIPOFECTAMINE® 2000, LIPOFECTAMINE® 3000, available from Thermo Fisher; VIAFECT™ Transfection Reagent, FUGENE® 6 Transfection Reagent, and FUGENE® HD Transfection Reagent, each of which is available from Promega Corporation; and TRANSFECTIN™ Lipid Reagent, available from BioRad Laboratories, Inc.

Additional exemplary transfection reagents include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, DMRIE-C, CELLFECTIN™ (Thermo Fisher Scientific), OLIGOFECTAMINE™ (Thermo Fisher Scientific), LIPOFECTACE™, TRANSFECTAM™ (Transfectam, Promega, Madison, Wis.), TFX-10™ (Promega), TFX-20™ (Promega), Tfx-50™ (Promega), SILENTFECT™ (Bio-Rad), EFFECTENE™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

In embodiments, the donor DNA is a single stranded DNA. In embodiments, the donor DNA is a double stranded DNA. In embodiments, the donor DNA is between 35 nucleotides and 10,000 nucleotides long. In embodiments, the donor DNA is between 50 nucleotides and 10,000 nucleotides long. In embodiments, the donor DNA is between 35 and 10,000 base pairs in length. In embodiments, the donor DNA is between 35 and 1,000 base pairs in length. In embodiments, the donor DNA is between 35 and 2,000 base pairs in length. In embodiments, the donor DNA is between 35 and 3,000 base pairs in length. In embodiments, the donor DNA is between 35 and 4,000 base pairs in length. In embodiments, the donor DNA is between 35 and 5,000 base pairs in length. In embodiments, the donor DNA is between 100 and 6,000 base pairs in length. In embodiments, the donor DNA is between 35 and 7,000 base pairs in length. In embodiments, the donor DNA is between 35 and 8,000 base pairs in length. In embodiments, the donor DNA is between 35 and 9,000 base pairs in length. In embodiments, the donor DNA is between 100 and 10,000 base pairs in length. In embodiments, the donor DNA is between 200 and 10,000 base pairs in length. In embodiments, the donor DNA is between 300 and 10,000 base pairs in length. In embodiments, the donor DNA is between 400 and 10,000 base pairs in length. In embodiments, the donor DNA is between 500 and 10,000 base pairs in length. In embodiments, the donor DNA is between 1,000 and 10,000 base pairs in length. In embodiments, the donor DNA is between 2,000 and 10,000 base pairs in length. In embodiments, the donor DNA is between 3,000 and 10,000 base pairs in length. In embodiments, the donor DNA is between 4,000 and 10,000 base pairs in length. In embodiments, the donor DNA is between 5,000 and 10,000 base pairs in length. In embodiments, the donor DNA is between 6,000 and 10,000 base pairs in length. In embodiments, the donor DNA is between 7,000 and 10,000 base pairs in length. In embodiments, the donor DNA is between 8,000 and 10,000 base pairs in length. In embodiments, the donor DNA is between 9,000 and 10,000 base pairs in length. The length of the donor DNA can be any value or subrange within the recited ranges, including endpoints.

In an aspect, a composition including cells, at least one non-specific single strand DNA binding protein, and at least one nucleic acid cutting entity is provided. In some embodiments, the composition further comprises a donor DNA.

In an aspect a kit for genetic modification is provided. In embodiments, the kit includes (i) a non-specific single strand DNA binding protein or nucleic acid encoding the at least one non-specific single stranded DNA binding protein; and (ii) a nucleic acid cutting entity or a nucleic acid encoding the nucleic acid cutting entity. In embodiments, the kit includes a transfection reagent. In embodiments, the kit includes a lipid transfection reagent. In embodiments, the kit includes a non-homologous end joining (NHEJ) inhibitor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Examples

One skilled in the art would understand that descriptions of making and using the particles described herein is for the sole purpose of illustration, and that the present disclosure is not limited by this illustration.

### Example 1. Effect of SSB on Editing Efficiency Using Short Single-Stranded Donor DNA

To determine the effect of single-stranded DNA binding proteins on gene HDR-mediated gene editing efficiency, Cas9 ribonucleoprotein (RNP) was delivered into 293FT cells with a ssDNA donor oligo (approx. 100 bases) in the presence of different single-stranded DNA binding proteins, using electroporation. The 293FT contain a disrupted Emerald Green Fluorescent Protein (EmGFP) gene; gene editing with the donor oligo restores EmGFP expression. In one 1.5 ml sterile Eppendorf tube, 1 μg of Cas9 protein was added to 3 μl of Resuspension Buffer R of NEON® transfection system (Thermo Fisher Scientific, cat. no. MPK1025), followed by addition of 240 ng gRNA targeting the disrupted EmGFP sequence (See wild type and disrupted EmGFP sequences in Table 2). The Cas9 and gRNA mixture was incubated at room temperature for 5 to 10 minutes to form the Cas9 RNP complexes. In a separate tube, 10 pmoles of phosphorothioate-modified ssDNA donor oligo (OEG GGT AGC GGG CGA AGC ACT GCA CGC CGT AGG TGA AGG TGG TCA CGA GGG TGG GCC AGG GCA CGG GCA GCT TGC CGG TGG TGC AGA TGA ACT TOF G (SEQ ID NO. 8)) were added to 3 μl of Resuspension Buffer R, followed by addition of various amount of single-stranded DNA binding protein (For example, 0 μg, 1.2 μg, 2.5 μg, and 5 μg of E. coli SSB were used).

For NLS-modified ssDNA donor oligo, 0.5 pmoles were used instead of 10 pmoles. The ssDNA donor oligo and single-stranded DNA binding protein were incubated at room temperature for 5-10 minutes to form the oligo/SSB complexes. The oligo/SSB complexes were then mixed with the Cas9 RNP complexes. Meanwhile, the disrupted EmGFP cells were detached from the culture flask and counted. Aliquot of cells were washed once with dPBS and cell pellet was re-suspended in Resuspension Buffer R at a cell density of 4×10⁷ cells/ml. Five microliter of cell suspension (2×10⁵ cells) was then transferred to the tube containing both Cas9 RNP and oligo/SSB complexes. Upon mixing, 10 μl of sample was used for electroporation using NEON® electroporation system with voltage set at 1150, pulse width set at 20 ms, and number of pulse set at 2. The electroporated cells were immediately transferred to a 24-well plate containing 0.5 ml of growth medium. At 48-72 hours post transfection, the cells were detached and analyzed by flow cytometry using ATTUNE® N×T Flow Cytometer instrument (Thermo Fisher Scientific). The percentage of EmGFP-positive cells were determined.

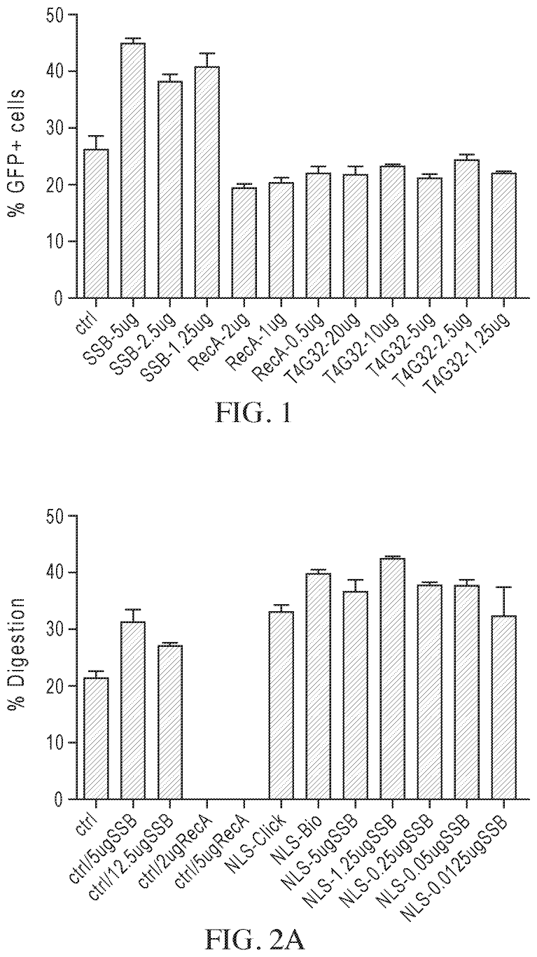

As shown in FIG. 1, the presence of E. coli SSB at any amount tested (5 μg, 2.5 μg, and 1.2 μg) increased the percentage of cells expressing EmGFP. Presence of RecA or T4G32 did not increase HDR-mediate gene editing efficiency.

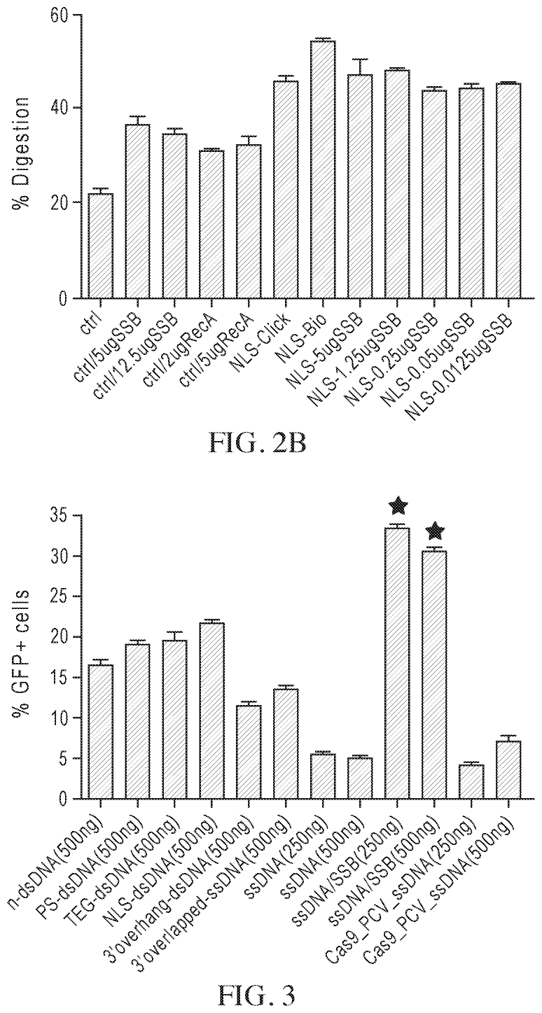

Next the effect of a ssDNA donor oligo attached to a nuclear localization signal (NLS) peptide, with or without SSB, on HDR-mediated gene editing efficiency was tested. 293FT cells were transfected with a ssDNA donor oligo (approx. 100 bases), with or without the NLS peptide, and Cas9 RNP in the presence of SSB using CRISPRMAX® transfection reagent (Thermo Fisher Scientific) (FIG. 2A); or with ssDNA donor oligo, with or without the NLS peptide, and Cas9 mRNA in the presence of SSB using MESSENGERMAX® transfection reagent (Thermo Fisher Scientific) (FIG. 2B).

As shown in FIGS. 2A and 2B, the presence of an NLS peptide on the donor oligo does not further increase HDR-mediated gene editing efficiency (shown as % digestion, representing the homology-directed repair efficiency because a restriction site is inserted near the Cas9 cleavage site) compared to SSB alone.

### Example 2. Effect of SSB on Editing Efficiency Using Long Single-Stranded Donor DNA

To determine the effect of SSB on HDR-mediated gene editing efficiency using a long (approx. 1.4 kb) donor DNA (see Table 2), Cas9 RNP was delivered into 293FT cells with a dsDNA donor, or a ssDNA donor in the presence or absence of SSB, using CRISPRMAX®. dsDNA donor was modified as follows: no modification (n); 5′ phosphorothioate (PS); 5′ tetraethylene glycol (TEG); 5′ NLS; 3′ overhang due to 5′ to 3′ nuclease digestion and precise termination with two internal consecutive phosphorothioate-modified nucleotides (3′ overhang). ssDNA donor was unmodified, 3′ overlapping due to prolonged 5′ to 3′ nuclease digestion; or attached to Cas9 RNP (Cas9_PCV). The percentage of cells expressing GFP was determined using flow cytometry.

As shown in FIG. 3, presence of SSB resulted in increased HDR mediated genome editing efficiency (shown as percentage of cells positive for GFP expression) compared to when a dsDNA donor (modified or unmodified) was used.

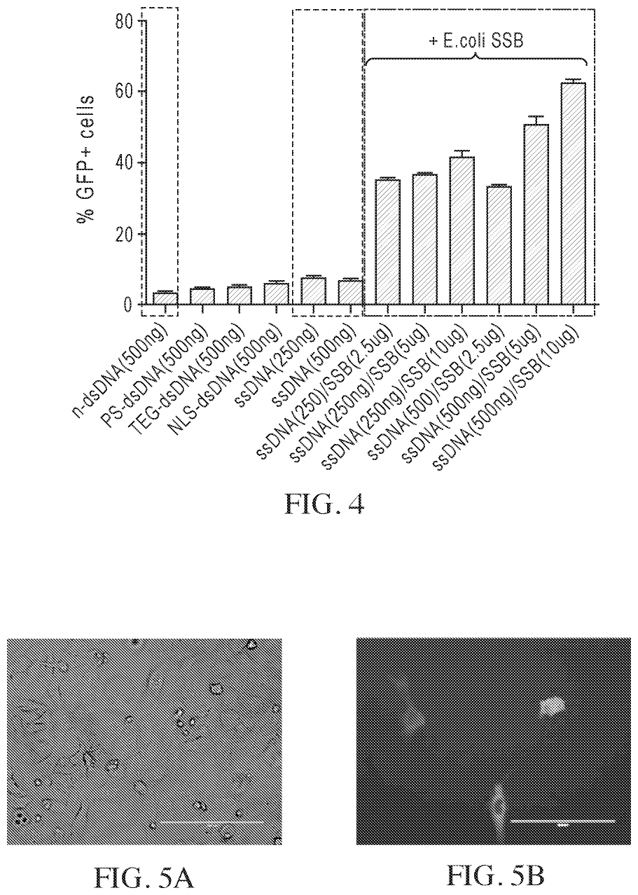

Similar results were observed in U2OS cells (FIG. 4). U2OS cells have very low HDR gene editing efficiency using dsDNA donor or ssDNA donor alone. The presence of E. coli SSB dramatically increased the number of GFP-expressing cells, regardless of the amount of ssDNA donor or SSB used.

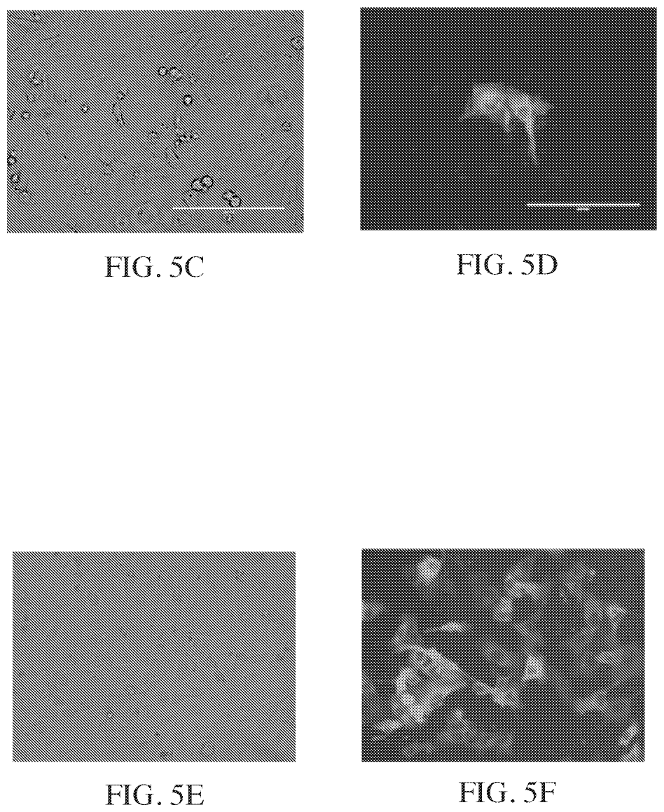

FIGS. 5A-5F show that transfection of U2OS cells with ssDNA donor in the presence of SSB results in increased GFP expression and fewer dead cells compared to 5′ PS modified dsDNA, or ssDNA alone.

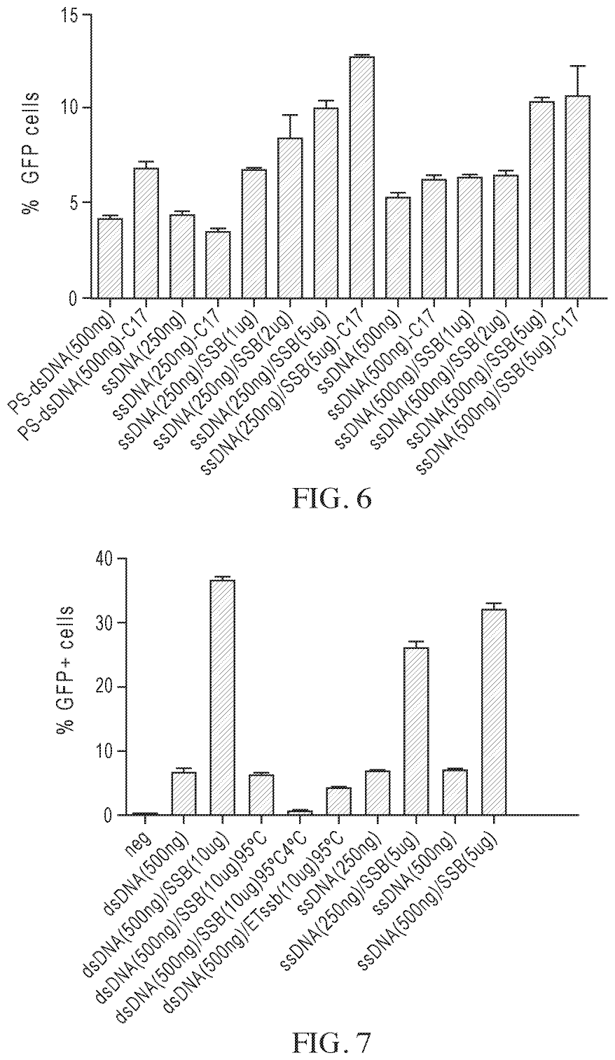

To determine whether treatment with a NHEJ inhibitor further increases HDR editing efficiency, U2OS cells were transfected with Cas9 RNP and 1.4 kb dsDNA or ssDNA donor, using NEON™ electroporation (Thermo Fisher Scientific). Treatment with C17 resulted in a slight increase in efficiency under some conditions (FIG. 6).

### Example 3. Effect of SSB on Editing Efficiency Using Long Double-Stranded Donor DNA

To determine whether SSB has an effect on HDR gene editing efficiency using a long (approx. 1.4 kb) double-stranded donor DNA, U2OS cells were transfected with Cas9 RNP and double-stranded donor DNA using CRISPRMAX®. dsDNA (500 ng)/SSB (10 μg): Unmodified double-stranded donor DNA (500 ng) was mixed with 10 μg E. coli SSB without heating. dsDNA (500 ng)/SSB (10 ug)95° C.: Unmodified double-stranded donor DNA (500 ng) was mixed with 10 μg E. coli SSB, heated at 95° C. for 10 minutes, then cooled to room temperature. dsDNA (500 ng)/SS (10 μg)95° C.4° C.: Unmodified double-stranded donor DNA (500 ng) was mixed with 10 μg E. coli SSB, heated at 95° C. for 10 minutes, then immediately put on ice. dsDNA (500 ng)/ETssb (10 ug) 95° C.: Unmodified double-stranded donor DNA (500 ng) was mixed with 10 μg thermostable SSB, heated at 95° C. for 10 minutes, then cooled to room temperature.

As shown in FIG. 7, E. coli SSB increased HDR-mediated editing efficiency when using a dsDNA donor, without heating.

### Example 4. Production of ssDNA Using Thermostable SSB

To make ssDNA, dsDNA is incubated at 95° C. for about 10 min. in the presence of thermostable SSB. The resulting ssDNA can be used directly in a gene editing protocol. Alternatively, one or each strand of the resulting ssDNA is purified. FIG. 8 shows an example protocol, with one strand of the dsDNA labeled with biotin (bio). The ssDNA is purified using streptavidin-coated beads and/or purified on a PCR column.

### Example 5. Production of ssDNA Using Sequential Exonuclease Digestion and Real-Time Monitoring

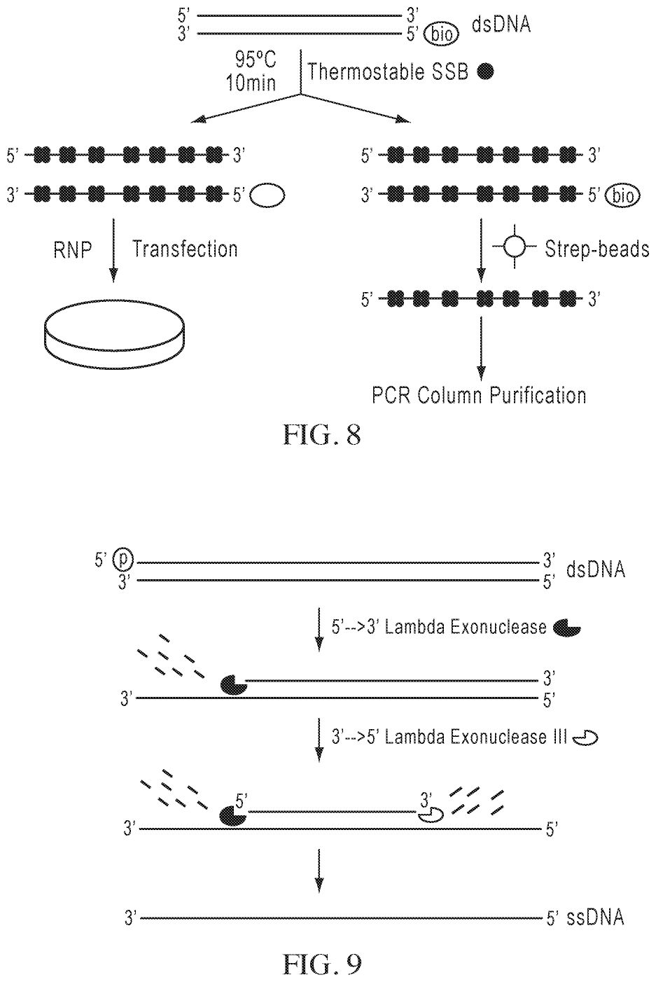

ssDNA was produced using a protocol as represented by FIG. 9, with or without a heat inactivation step. For the heat-inactivation protocol, dsDNA having one strand modified with a 5′ phosphate was incubated with lambda exonuclease (a 5′ to 3′ exonuclease that requires the 5′ phosphate modification) in 10× λExo buffer (670 mM Glycine-KOH, pH 9.4, 25 mM MgCl₂, 0.1% v/v TRITON™ X-100) at 37° C. for 5 min/kb (e.g., about 7 min. for 1.4 kb dsDNA, about 10 min for 4.2 kb) with PICOGREEN® dye (1:20,000 dilution) (Thermo Fisher Scientific, Waltham, Mass.). PICOGREEN® dye binds preferentially to dsDNA, not ssDNA. Progression of the digestion (based on PICOGREEN® dye fluorescence in the sample) was continuously monitored by QUBIT™ Fluorometric Quantification (Thermo Fisher Scientific). The lambda exonuclease was heat killed at 80° C. for 5-10 min. The DNA was then incubated with exonuclease III (a 3′ to 5′ dsDNA exonuclease) in 2× ExoIII buffer (660 mM Tris-HCl, pH 8.0, 6.6 mM MgCl₂) with PICOGREEN® at 37° C. for 5 min/kb. Progression of the digestion was continuously monitored by QUBIT™ Fluorometric Quantification) (Thermo Fisher Scientific, Waltham, Mass.). The exonuclease was heat killed at 80° C. for 5-10 min. See, e.g., U.S. Patent Pub. 2017/0349927, which is incorporated herein by reference in its entirety. The resulting DNA was purified on a PCR column, and DNA concentration was measured by nanodrop.

For the protocol without heat inactivation, dsDNA having one strand modified with a 5′ phosphate was incubated with lambda exonuclease at 37° C. in 10× TrueString buffer (660 mM Tris-HCl, pH 8.5, 10 to 50 mM MgCl₂, 0.1% (v/v) TRITON™ X-100) with PICOGREEN® dye (Thermo Fisher Scientific, Waltham, Mass.) (1:2,000 dilution). Progression of the digestion was continuously monitored by QUBIT™ Fluorometric Quantification until the amount of dsDNA plateaued. At plateau, exonuclease III was added, without changing the buffer or temperature, and incubated at 37° C. Progression of the digestion was continuously monitored by QUBIT™ Fluorometric Quantification (Thermo Fisher Scientific, Waltham, Mass.) until the amount of dsDNA plateaued. Stop buffer can optionally be added at plateau. The resulting DNA was purified on a PCR column, and DNA concentration was measured by nanodrop.

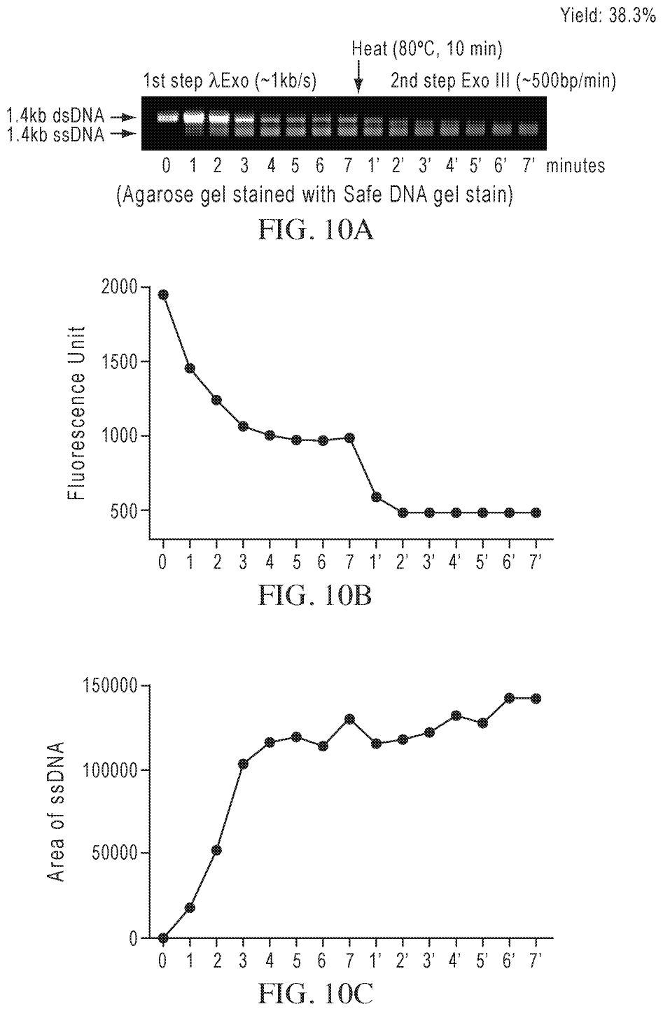

FIG. 10A is a photograph of an agarose gel, showing the kinetics over time of the heat inactivation protocol, starting with a 1.4 kb dsDNA. Gel was stained with SYBR™ Safe DNA Gel Stain. The yield from this method was 38.3%. FIG. 10B shows the kinetics as measured by QUBIT™ Fluorometric Quantification. FIG. 10C shows the kinetics as measured by area of ssDNA, shown in the bottom band of FIG. 10A.

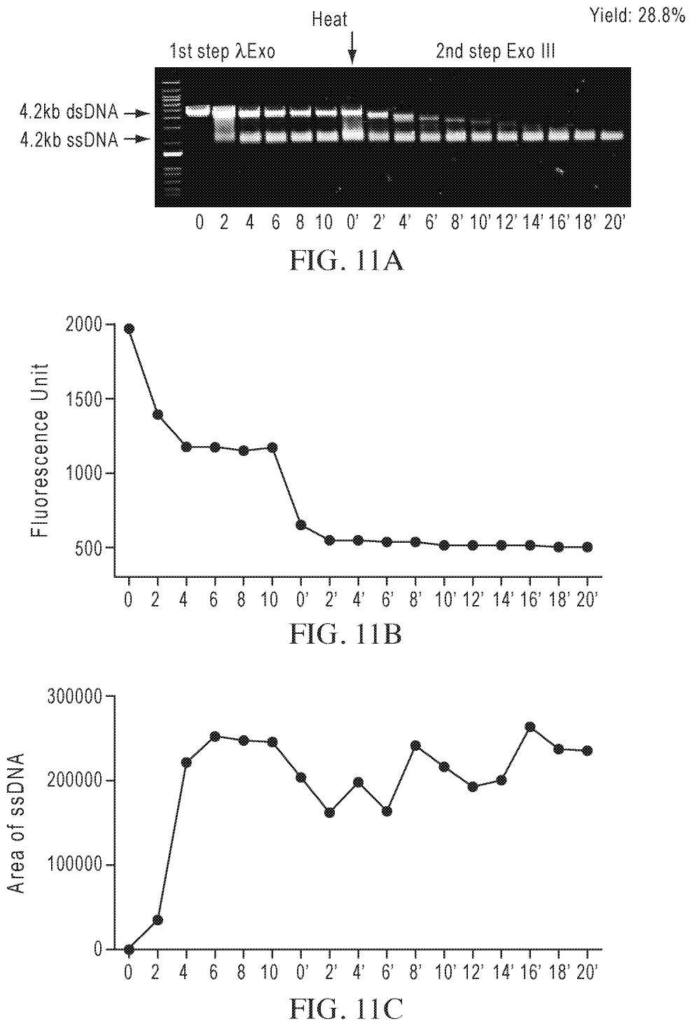

FIG. 11A is a photograph of an agarose gel, showing the kinetics over time of the heat inactivation protocol, starting with a 4.2 kb dsDNA. Gel was stained with SYBR™ Safe DNA Gel Stain. The yield from this method was 28.8%. FIG. 11B shows the kinetics as measured by QUBIT™ Fluorometric Quantification. FIG. 11C shows the kinetics as measured by area of ssDNA, shown in the bottom band of FIG. 11A.

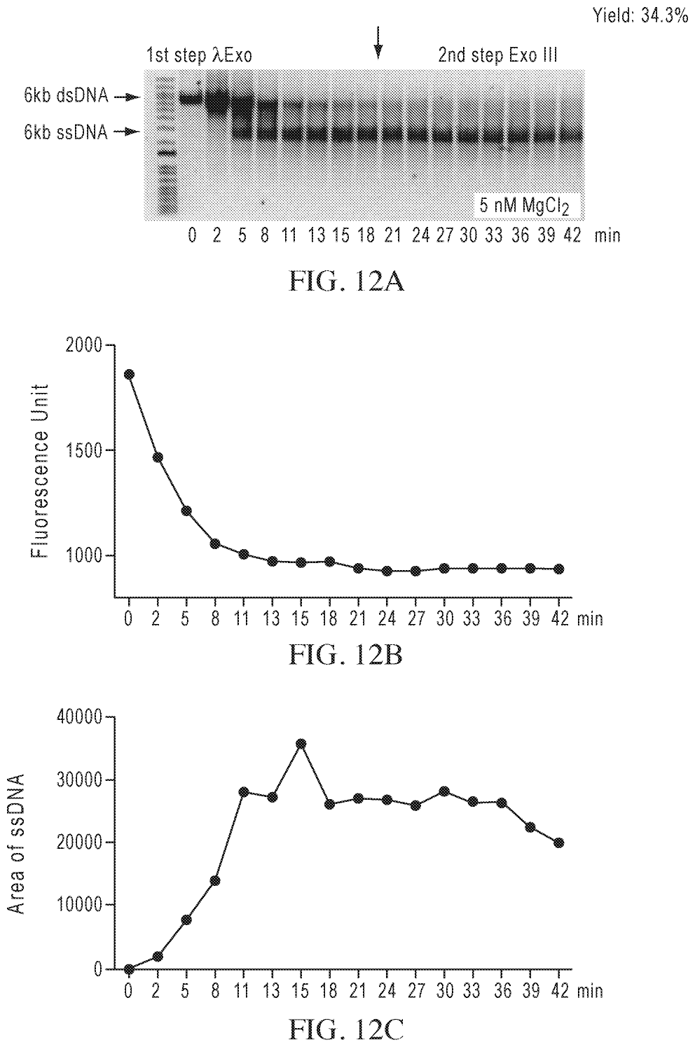

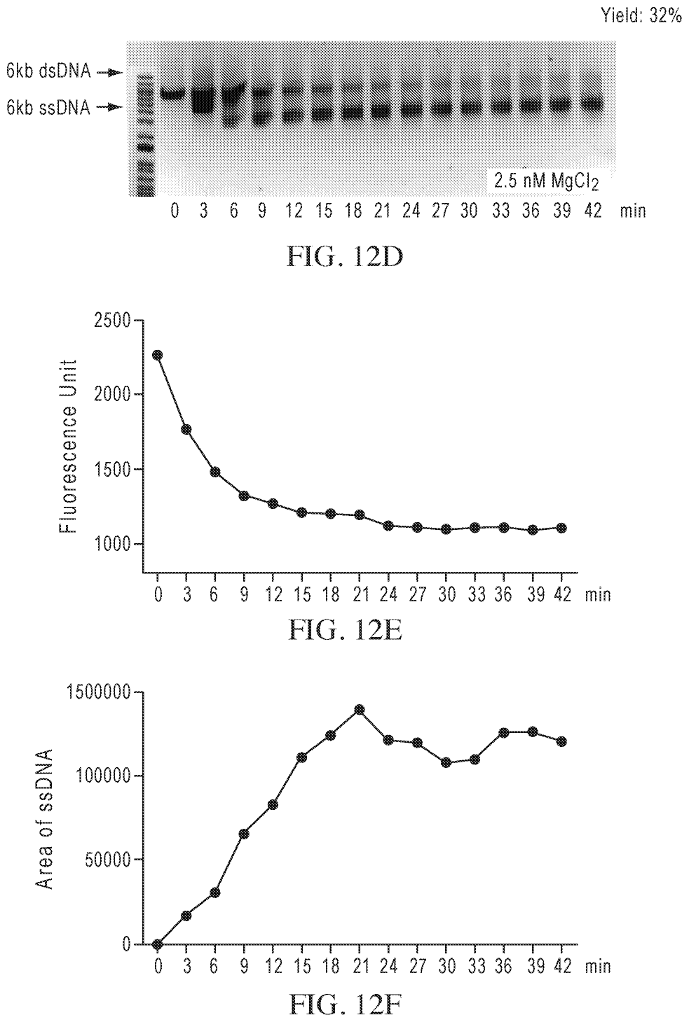

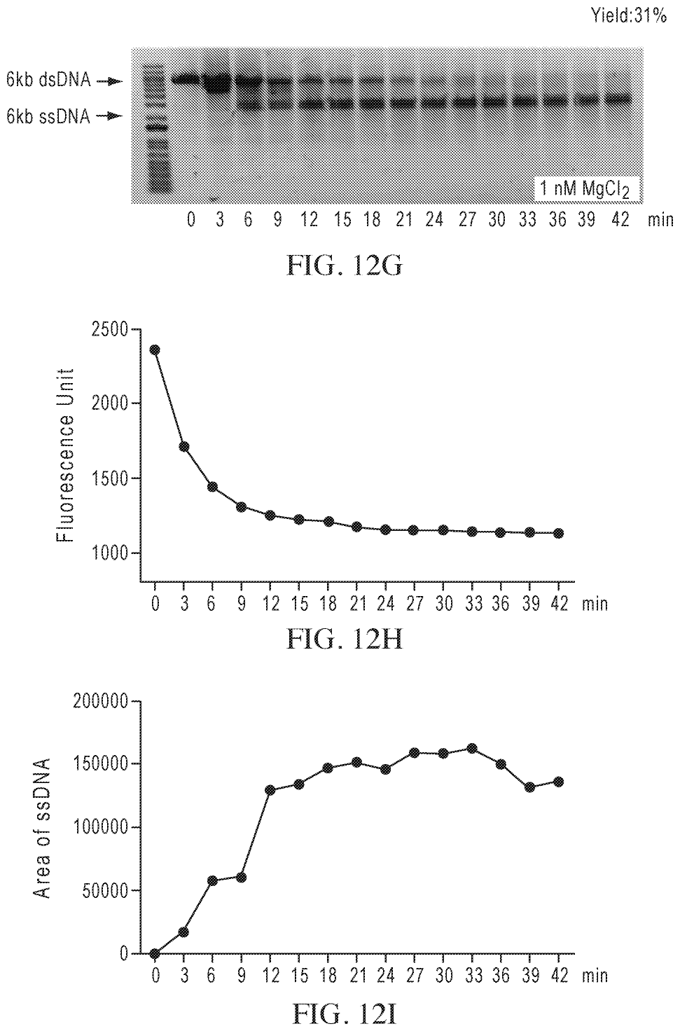

FIGS. 12A-12I shows the kinetics over time of the protocol without heat inactivation, starting with a 6 kb dsDNA. Digestion was performed in the presence of 5 mM MgCl₂ (FIGS. 12A-12C), 2.5 mM MgCl₂ (FIG. 12D-12F), or 1 mM MgCl₂ (FIGS. 12G-12I). ssDNA production was monitored by agarose gel staining with SYBR™ Safe DNA Gel Stain (top row), QUBIT™ Fluorometric Quantification (middle row), or area of ssDNA in the agarose gels (bottom row). The yield from this method was 34.3%, 32%, and 31% at 5 mM, 2.5 mM, and 1 mM MgCl₂, respectively. Increasing MgCl₂ concentration also sped up the reaction.

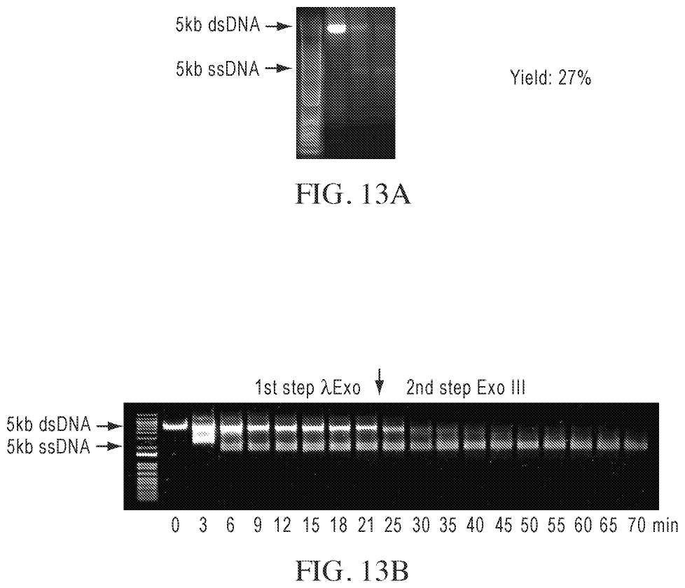

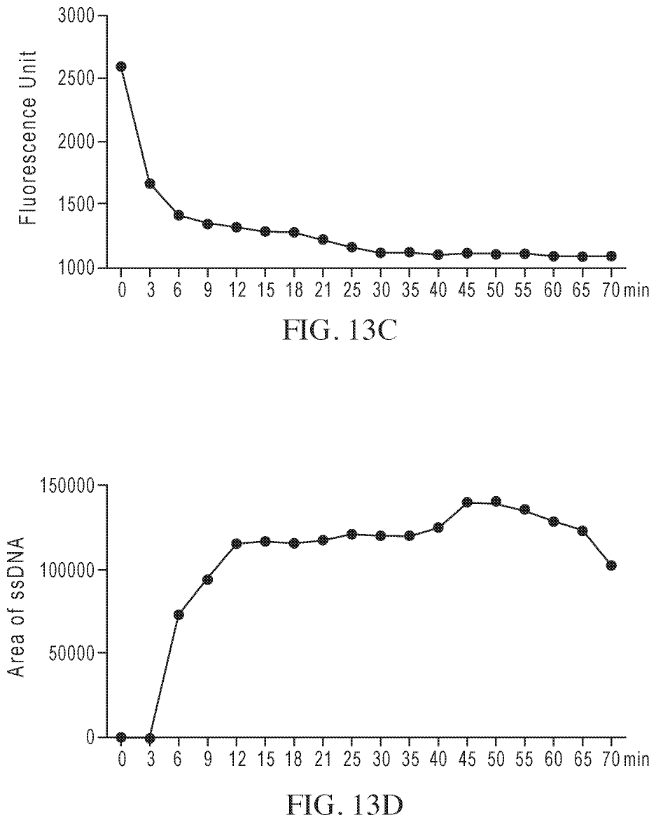

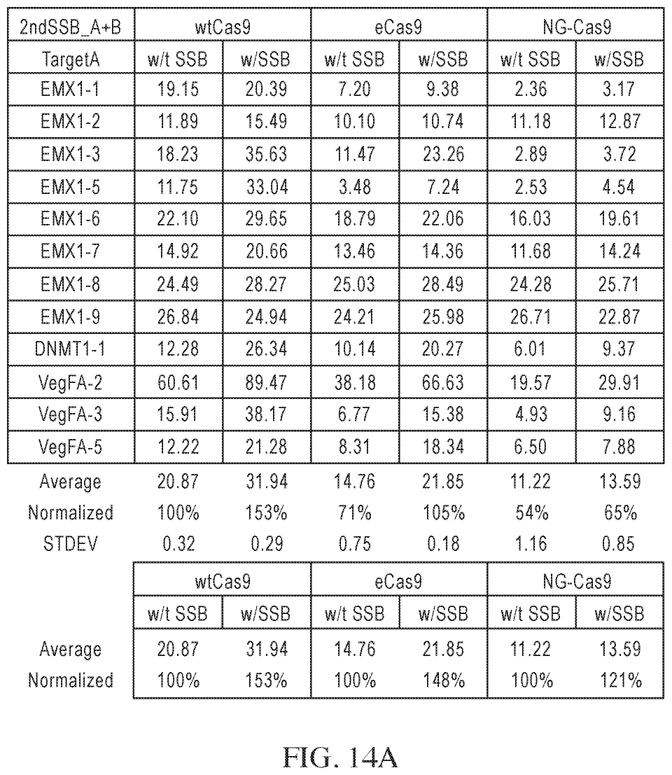

FIG. 13A shows relative levels of starting dsDNA (second column from left) and resulting dsDNA and ssDNA using the heat-inactivation protocol on a 5 kb starting dsDNA template. FIG. 13B shows the kinetics over time of the protocol without heat inactivation on the 5 kb starting dsDNA template. The yield using the protocol without heat inactivation was 27% at 70 min., but would have been higher if the reaction had been stopped sooner.

### Example 6. Effect of SSB on NHEJ Editing Efficiency at Different Targets

To determine whether the presence of SSB affects NHEJ editing efficiency. The wild type Cas9 protein (0.2 μg), or other Cas9 variants was mixed with gRNA (1.5 pmol) for 2-3 minutes, then mixed with or without E. coli SSB (1 μg) (Thermo Fisher Scientific, cat. no. 70032Z500UG), for another 2-3 minutes and delivered into 293FT cells (15,000 cells) using CRISPRMAX® transfection reagent (Thermo Fisher Scientific). Three versions of Cas9 were tested: wild type (wtCas9), high fidelity Cas9 (eCas9), and NG-Cas9, which has a NG protospacer adjacent motif (PAM) instead of NGG PAM. The wtCas9 was the TRUECUT™ Cas9 v2 protein (Thermo Fisher Scientific, cat. no. A36496). The high fidelity eCas9 was also referred to as eSpCas9(1.1), which contains three amino acid mutations (K848A/K1003A/R1060A) to the wild-type Cas9. The NG-Cas9 was also referred to as SpCas9-NG, which contains seven amino acid mutations (A262T/R324L/5409I/E480K/E543D/M694I/E1219V) to the wild-type Cas9. Twelve different targets were tested (FIGS. 14 and 15). Cells were analyzed by next-generation sequencing (NGS) targeted Amplicon-seq after 3 days. NGS Targeted Amplicon-seq was performed by: PCR amplifying the edited region in 200-400 bp with the cleavage site in the middle; ligating a NGS (Ion Torrent or Illumina) barcoded adaptor to amplicons; pooling barcoded samples and sequencing them using NGS. The percentage of edited (cleavage) and non-edited sequences (reads) were calculated.

NHEJ-mediated genome editing efficiency was improved regardless of target or Cas9 used (FIGS. 14A and 14B). Y-axis label in FIG. 14B is the normalized average edited sequence % compared to the wtCas9. The numbers in FIG. 14A represent the edited sequence % from “NGS Targeted Amplicon-seq” for each sample.

As shown in FIG. 15, using SSB as a supplement to eCas9 improves the lower NHEJ editing efficiency of eCas9 to near wild type levels across multiple targets.

TABLE 2
Nucleotide Sequences

| | SEQ ID |
|---|---|
| Sequence Title/Nucleotide Sequence | NO |
| ssDNA Donor Oligo Sequence: | 8 |
| OEG GGT AGC GGG CGA AGC ACT GCA CGC CGT AGG TGA AGG TGG TCA | |
| CGA GGG TGG GCC AGG GCA CGG GCA GCT TGC CGG TGG TGC AGA TGA | |
| ACT TOF G | |
| F: Phosphorothioate-A; O: Phosphorothioate-C; | |
| E: Phosphorothioate-G; Z: Phosphorothioate-T | |
| 1.4 kb Single-Stranded or Double-Stranded Donor DNA Sequence: | 9 |
| [symbol] ATGGGAGGTAAGCCCTTGCATTCGA | |
| CCGAGTACAAGCCCACAGTGCGGCTGGCCACCAGGGACGATGTGCCTAGAGCTGTGC | |
| GGACACTGGCCGCTGCCTTCGCCGATTACCCTGCCACCAGACACACCGTGGACCCCG | |
| ACAGACACATCGAGAGAGTGACCGAGCTGCAGGAACTGTTTCTGACCAGAGTGGGCC | |
| TGGACATCGGCAAAGTGTGGGTGGCCGATGATGGCGCCGCTGTGGCTGTGTGGACAA | |
| CCCCTGAGTCTGTGGAAGCCGGCGCTGTGTTCGCCGAGATCGGACCTAGAATGGCCG | |
| AGCTGAGCGGCTCTAGACTGGCTGCCCAGCAGCAGATGGAAGGCCTGCTGGCCCCCC | |
| ACAGACCTAAAGAGCCTGCCTGGTTTCTGGCCACCGTGGGCGTGTCACCTGACCACC | |
| AGGGCAAGGGACTGGGATCTGCTGTGGTGCTGCCTGGCGTGGAAGCTGCTGAAAGGG | |
| CTGGCGTGCCCGCCTTCCTGGAAACAAGCGCCCCCAGAAACCTGCCCTTCTACGAGA | |
| GACTGGGCTTCACCGTGACCGCCGACGTGGAAGTGCCTGAGGGCCCTAGAACCTGGT | |
| GCATGACCAGAAAGCCTGGCGCCGGAAGCGGAGCTACTAACTTCAGCCTGCTGAAGC | |
| AGGCTGGAGACGTGGAGGAGAACCCTGGACCTGTGAGCAAGGGCGAGGAGCTGTTCA | |
| CCGGGGTGGTGCCCATCCTGGTCGAGCTGGACGGCGACGTAAACGGCCACAAGTTCA | |
| GCGTGTCCGGCGAGGGCGAGGGCGATGCCACCTACGGCAAGCTGACCCTGAAGTTCA | |
| TCTGCACCACCGGCAAGCTGCCCGTGCCCTGGCCCACCCTCGTGACCACCTTCACCT | |
| ACGGCGTGCAGTGCTTCGCCCGCTACCCCGACCACATGAAGCAGCACGACTTCTTCA | |
| AGTCCGCCATGCCCGAAGGCTACGTCCAGGAGCGCACCATCTTCTTCAAGGACGACG | |
| GCAACTACAAGACCCGCGCCGAGGTGAAGTTCGAGGGCGACACCCTGGTGAACCGCA | |
| TCGAGCTGAAGGGCATCGACTTCAAGGAGGACGGCAACATCCTGGGGCACAAGCTGG | |
| AGTACAACTACAACAGCCACAAGGTCTATATCACCGCCGACAAGCAGAAGAACGGCA | |
| TCAAGGTGAACTTCAAGACCCGCCACAACATCGAGGACGGCAGCGTGCAGCTCGCCG | |
| ACCACTACCAGCAGAACACCCCCATCGGCGACGGCCCCGTGCTGCTGCCCGACAACC | |
| ACTACCTGAGCACCCAGTCCGCCCTGAGCAAAGACCCCAACGAGAAGCGCGATCACA | |
| TGGTCCTGCTGGAGTTCGTGACCGCCGCCGGGATCACTCTCGGCATGGACGAGCTGT | |
| ACAAGGGTTCAGGTAGTGGAAGCGGT [symbol] | |
| [symbol] | |
| Note: Homology arm sequences are shown with bold, underlining | |
| Wild type EmGFP Sequence: | 10 |
| ATGGTGAGCAAGGGCGAGGAGCTGTTCACCGGGGTGGTGCCCATCCTGGTCGAGCTG | |
| GACGGCGACGTAAACGGCCACAAGTTCAGCGTGTCCGGCGAGGGCGAGGGCGATGCC | |
| ACCTACGGCAAGCTGACCCTGAAGTTCATCTGCACCACCGGCAAGCTGCCCGTGCCC | |
| TGGCCCACCCTCGTGACCACCTTCACCTACGGCGTGCAGTGCTTCGCCCGCTACCCC | |
| GACCACATGAAGCAGCACGACTTCTTCAAGTCCGCCATGCCCGAAGGCTACGTCCAG | |
| GAGCGCACCATCTTCTTCAAGGACGACGGCAACTACAAGACCCGCGCCGAGGTGAAG | |
| TTCGAGGGCGACACCCTGGTGAACCGCATCGAGCTGAAGGGCATCGACTTCAAGGAG | |
| GACGGCAACATCCTGGGGCACAAGCTGGAGTACAACTACAACAGCCACAAGGTCTAT | |
| ATCACCGCCGACAAGCAGAAGAACGGCATCAAGGTGAACTTCAAGACCCGCCACAAC | |
| ATCGAGGACGGCAGCGTGCAGCTCGCCGACCACTACCAGCAGAACACCCCCATCGGC | |
| GACGGCCCCGTGCTGCTGCCCGACAACCACTACCTGAGCACCCAGTCCGCCCTGAGC | |
| AAAGACCCCAACGAGAAGCGCGATCACATGGTCCTGCTGGAGTTCGTGACCGCCGCC | |
| GGGATCACTCTCGGCATGGACGAGCTGTACAAGTAA | |
| Disrupted EmGFP Sequence: | 11 |
| ATGGTGAGCAAGGGCGAGGAGCTGTTCACCGGGGTGGTGCCCATCCTGGTCGAGCTG | |
| GACGGCGACGTAAACGGCCACAAGTTCAGCGTGTCCGGCGAGGGCGAGGGCGATGCC | |
| ACCTACGGCAAGCTGACCCTGAAGTTCATCTGCACCACCGGCAAGCTGCCCGTGCCC | |
| TGGCCCACCCTCGTGACCACC [symbol] TACGGCGTGCAGTGCTTCGCCCGCTACCCC | |
| GACCACATGAAGCAGCACGACTTCTTCAAGTCCGCCATGCCCGAAGGCTACGTCCAG | |
| GAGCGCACCATCTTCTTCAAGGACGACGGCAACTACAAGACCCGCGCCGAGGTGAAG | |
| TTCGAGGGCGACACCCTGGTGAACCGCATCGAGCTGAAGGGCATCGACTTCAAGGAG | |
| GACGGCAACATCCTGGGGCACAAGCTGGAGTACAACTACAACAGCCACAAGGTCTAT | |
| ATCACCGCCGACAAGCAGAAGAACGGCATCAAGGTGAACTTCAAGACCCGCCACAAC | |
| ATCGAGGACGGCAGCGTGCAGCTCGCCGACCACTACCAGCAGAACACCCCCATCGGC | |
| GACGGCCCCGTGCTGCTGCCCGACAACCACTACCTGAGCACCCAGTCCGCCCTGAGC | |
| AAAGACCCCAACGAGAAGCGCGATCACATGGTCCTGCTGGAGTTCGTGACCGCCGCC | |
| GGGATCACTCTCGGCATGGACGAGCTGTACAAGTAA | |
| Note: The delete sequence is shown with bold, strikethrough | |

TABLE 3
Exemplary Single-Stranded Binding Proteins

| | SEQ ID |
|---|---|
| Protein Designation/Amino Acid Sequence | NO |
| E. coli T4 virus gp32 (T4G32) single-stranded DNA binding protein | 12 |
| MFKRKSTAEL AAQMAKLNGN KGFSSEDKGE WKLKLDNAGN GQAVIRFLPS KNDEQAPFAI | |
| LVNHGFKKNG KWYIETCSST HGDYDSCPVC QYISKNDLYN TDNKEYSLVK RKTSYWANIL | |
| VVKDPAAPEN EGKVFKYRFG KKIWDKINAM IAVDVEMGET PVDVTCPWEG ANFVLKVKQV | |
| SGFSNYDESK FLNQSAIPNI DDESFQKELF EQMVDLSEMT SKDKFKSFEE LNTKFGQVMG | |
| TAVMGGAAAT AAKKADKVAD DLDAFNVDDF NTKTEDDFMS SSSGSSSSAD DTDLDDLLND | |
| L | |
| E. coli T4 virus uvsX protein | 13 |
| MSVLEKLKKN STLKTTAVLS KSSFFNEKTN TRTKIPMLNI AFSGDLKKGF QSGLIFFAGP | |
| SKHFKSNMGL TCVSAYMKQN PDAACLFFDS EFGITPAYLE SMGVDPDRVV HVPIKNIEEL | |
| KFEIMNQLEQ ITREDKVIIF IDSIGNLASK KEVEDAINEK SAQDMTRAKA LKGLFRMVTP | |
| YLTMNDIPCI AINHTYETQE MFSKTVMSGG TGAMYSANEV FIIGRRQQKE GTEITGYDFI | |
| LNAEKSRTVK EKSKFPISVT FSGGIDPYSG LLELAVELGW VVKPSNGWYS RSILNTETGE | |
| METEERKFRA KETNSIEFWK PLLTNDKFNE AINDHYKLGQ VISDEAVDKE IEDMLA | |
| E. coli T4 virus uvsY protein | 14 |
| MDLNDLKEQL EADMKIDATK LQWEALNNPV VYSKWLRIYS EAKRETIALE AKKKKAMKNR | |
| LDFYTNRSDD WCRAEYEKSE LKVVMAADDE ILPLDTKIAY YQMVMDFAGR ALDIVKSRGF | |
| AIKNAIELRM LESGR | |
| Bloom syndrome protein | 15 |
| MAAVPQNNLQ EQLERHSART LNNKLSLSKP KFSGFTFKKK TSSDNNVSVT NVSVAKTPVL | |
| RNKDVNVTED FSFSEPLPNT TNQQRVKDFF KNAPAGQETQ RGGSKSLLPD FLQTPKEVVC | |
| TTQNTPTVKK SRDTALKKLE FSSSPDSLST INDWDDMDDF DTSETSKSFV TPPQSHFVRV | |
| STAQKSKKGK RNFFKAQLYT TNTVKTDLPP PSSESEQIDL TEEQKDDSEW LSSDVICIDD | |
| GPIAEVHINE DAQESDSLKT HLEDERDNSE KKKNLEEAEL HSTEKVPCIE FDDDDYDTDF | |
| VPPSPEEIIS ASSSSSKCLS TLKDLDTSDR KEDVLSTSKD LLSKPEKMSM QELNPETSTD | |
| CDARQISLQQ QLIHVMEHIC KLIDTIPDDK LKLLDCGNEL LQQRNIRRKL LTEVDFNKSD | |
| ASLLGSLWRY RPDSLDGPME GDSCPTGNSM KELNFSHLPS NSVSPGDCLL TTTLGKTGFS | |
| ATRKNLFERP LFNTHLQKSF VSSNWAETPR LGKKNESSYF PGNVLTSTAV KDQNKHTASI | |
| NDLERETQPS YDIDNFDIDD FDDDDDWEDI MHNLAASKSS TAAYQPIKEG RPIKSVSERL | |
| SSAKTDCLPV SSTAQNINFS ESIQNYTDKS AQNLASRNLK HERFQSLSFP HTKEMMKIFH | |
| KKFGLHNFRT NQLEAINAAL LGEDCFILMP TGGGKSLCYQ LPACVSPGVT VVISPLRSLI | |
| VDQVQKLTSL DIPATYLTGD KTDSEATNIY LQLSKKDPII KLLYVTPEKI CASNRLISTL | |
| ENLYERKLLA RFVIDEAHCV SQWGHDFRQD YKRMNMLRQK FPSVPVMALT ATANPRVQKD | |
| ILTQLKILRP QVFSMSFNRH NLKYYVLPKK PKKVAFDCLE WIRKHHPYDS GIIYCLSRRE | |
| CDTMADTLQR DGLAALAYHA GLSDSARDEV QQKWINQDGC QVICATIAFG MGIDKPDVRF | |
| VIHASLPKSV EGYYQESGRA GRDGEISHCL LFYTYHDVTR LKRLIMMEKD GNHHTRETHF | |
| NNLYSMVHYC ENITECRRIQ LLAYFGENGF NPDFCKKHPD VSCDNCCKTK DYKTRDVTDD | |
| VKSIVRFVQE HSSSQGMRNI KHVGPSGRFT MNMLVDIFLG SKSAKIQSGI FGKGSAYSRH | |
| NAERLFKKLI LDKILDEDLY INANDQAIAY VMLGNKAQTV LNGNLKVDFM ETENSSSVKK | |
| QKALVAKVSQ REEMVKKCLG ELTEVCKSLG KVFGVHYFNI FNTVTLKKLA ESLSSDPEVL | |
| LQIDGVTEDK LEKYGAEVIS VLQKYSEWTS PAEDSSPGIS LSSSRGPGRS AAEELDEEIP | |
| VSSHYFASKT RNERKRKKMP ASQRSKRRKT ASSGSKAKGG SATCRKISSK TKSSSIIGSS | |
| SASHTSQATS GANSKLGIMA PPKPINRPFL KPSYAFS | |
| Rad51 Protein | 16 |
| MAMQMQLEAN ADTSVEEESF GPQPISRLEQ CGINANDVKK LEEAGFHTVE AVAYAPKKEL | |
| INIKGISEAK ADKILAEAAK LVPMGFTTAT EFHQRRSEII QITTGSKELD KLLQGGIETG | |
| SITEMFGEFR TGKTQICHTL AVTCQLPIDR GGGEGKAMYI DTEGTFRPER LLAVAERYGL | |
| SGSDVLDNVA YARAFNTDHQ TQLLYQASAM MVESRYALLI VDSATALYRT DYSGRGELSA | |
| RQMHLARFLR MLLRLADEFG VAVVITNQVV AQVDGAAMFA ADPKKPIGGN IIAHASTTRL | |
| YLRKGRGETR ICKIYDSPCL PEAEAMFAIN ADGVGDAKD | |
| Topoisomerase I | 17 |
| MSGDHLHNDS QIEADFRLND SHKHKDKHKD REHRHKEHKK EKDREKSKHS NSEHKDSEKK | |
| HKEKEKTKHK DGSSEKHKDK HKDRDKEKRK EEKVRASGDA KIKKEKENGF SSPPQIKDEP | |
| EDDGYFVPPK EDIKPLKRPR DEDDADYKPK KIKTEDTKKE KKRKLEEEED GKLKKPKNKD | |
| KDKKVPEPDN KKKKPKKEEE QKWKWWEEER YPEGIKWKFL EHKGPVFAPP YEPLPENVKF | |
| YYDGKVMKLS PKAEEVATFF AKMLDHEYTT KEIFRKNFFK DWRKEMTNEE KNIITNLSKC | |
| DFTQMSQYFK AQTEARKQMS KEEKLKIKEE NEKLLKEYGF CIMDNHKERI ANFKIEPPGL | |
| FRGRGNHPKM GMLKRRIMPE DIIINCSKDA KVPSPPPGHK WKEVRHDNKV TWLVSWTENI | |
| QGSIKYIMLN PSSRIKGEKD WQKYETARRL KKCVDKIRNQ YREDWKSKEM KVRQRAVALY | |
| FIDKLALRAG NEKEEGETAD TVGCCSLRVE HINLHPELDG QEYVVEFDFL GKDSIRYYNK | |
| VPVEKRVFKN LQLFMENKQP EDDLFDRLNT GILNKHLQDL MEGLTAKVFR TYNASITLQQ | |
| QLKELTAPDE NIPAKILSYN RANRAVAILC NHQRAPPKTF EKSMMNLQTK IDAKKEQLAD | |
| ARRDLKSAKA DAKVMKDAKT KKVVESKKKA VQRLEEQLMK LEVQATDREE NKQIALGTSK | |
| LNYLDPRITV AWCKKWGVPI EKIYNKTQRE KFAWAIDMAD EDYEF | |
| Vaccinia virus DNA topoisomerase type IB | 18 |
| MRALFYKDGK LFTDNNFLNP VLDDNPAYEV LQHVKIPTHL TDVVVYEQTW EEALTRLIFV | |
| GSDSKGRRQY FYGKMHVQNR NAKRDRIFVR VYNVMKRINC FINKNIKKSS TDSNYQLAVF | |
| MLMETMFFIR FGKMKYLKEN ETVGLLTLKN KHIEISPDEI VIKFVGKDKV SHEFVVHKSN | |
| RLYKPLLKLT DDSSPEEFLF NKLSERKVYE CIKQFGIRIK DLRTYGVNYT FLYNFWTNVK | |
| SISPLPSPKK LIALTIKQTA EVVGHTPSIS KRAYMATTIL EMVKDKNFLD VVSKTTFDEF | |
| LSIVVDHVKS STDG | |

The invention is further represented by the following clauses:

Clause 1. A method for preparing single stranded DNA (ssDNA), comprising: (a) providing a composition comprising a double stranded DNA (dsDNA) having a modification on the 5′ end of one strand; (b) contacting the composition with a lambda exonuclease; and (c) contacting the composition comprising the lambda exonuclease with exonuclease III or T7 exonuclease; wherein step (b) and step (c) are performed without changing buffer; thereby preparing ssDNA.

Clause 2. A method for preparing single stranded DNA (ssDNA), comprising: (a) providing a composition comprising a double stranded DNA (dsDNA) having a modification on the 5′ end of one strand; (b) contacting the composition with a lambda exonuclease; and (c) contacting the composition comprising the lambda exonuclease with exonuclease III or T7 exonuclease; wherein step (c) is performed without inactivating the lambda exonuclease; thereby preparing ssDNA.

Clause 3. The method of clause 1 or 2, wherein the 5′ modification is a 5′ phosphate.

Clause 4. The method of any one of clauses 1 to 3, wherein the composition further comprises a detectable marker that binds to a nucleotide.

Clause 5. The method of clause 4, wherein the detectable marker is a fluorescent nucleic acid stain.

Clause 6. The method of clause 5, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

Clause 7. The method of any one of clauses 4 to 6, further comprising monitoring an amount of detectable marker in the composition.

Clause 8. The method of clause 7, wherein the exonuclease III is added when the amount of the detectable marker reaches a plateau.

Clause 9. The method of any one of clauses 1 to 8, wherein the temperature of the composition does not exceed about 50° C.

Clause 10. The method of clause 9, wherein the temperature of the composition is maintained between about 20° C. and about 50° C.

Clause 11. The method of clause 9, wherein the temperature of the composition is maintained between about 30° C. and about 40° C.

Clause 12. The method of clause 9, wherein the temperature of the composition is maintained at about 37° C.

Clause 13. The method of one of clauses 1 to 12, further comprising adding a stop buffer.

Clause 14. The method of one of clauses 1 to 13, further comprising purifying the ssDNA.

Clause 15. The method of any one of clauses 1 to 14, wherein the modification comprises a phosphate.

Clause 16. The method of any one of clauses 1 to 15, wherein the dsDNA is between 100 and 10,000 base pairs in length.

Clause 17. The method of any one of clauses 1 to 16, wherein the composition comprises a magnesium salt.

Clause 18. The method of clause 17, wherein the composition comprises between 1 mM and 10 mM MgCl₂.

Clause 19. A composition comprising dsDNA, a lambda exonuclease, exonuclease III, and a detectable marker that binds to nucleotides.

Clause 20. The composition of clause 19, further comprising a magnesium salt.

Clause 21. The composition of clause 19 or 20, wherein the detectable marker is a fluorescent nucleic acid stain.

Clause 22. The composition of clause 21, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

Clause 23. A kit comprising a lambda exonuclease, exonuclease III, and a detectable marker that binds to nucleotides.

Clause 24. The kit of clause 23, further comprising a magnesium salt.

Clause 25. The kit of clause 23 or 24, wherein the detectable marker is a fluorescent nucleic acid stain.

Clause 26. The kit of clause 25, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

Clause 27. The kit of any one of clauses 23 to 26, further comprising a reaction buffer, wherein the reaction buffer is compatible with lambda exonuclease and exonuclease III.

Clause 28. A system comprising: (a) a device configured to detect a detectable marker that binds to a nucleotide; and (b) a composition comprising DNA, an exonuclease, and a detectable marker that binds to nucleotides.

Clause 29. The system of clause 28, wherein the exonuclease comprises a lambda exonuclease and/or exonuclease III.

Clause 30. The system of clause 28, wherein the exonuclease comprises a lambda exonuclease and exonuclease III.

Clause 31. The system of any one of clauses 28 to 30, wherein the detectable marker is a fluorescent nucleic acid stain.

Clause 32. The system of clause 31, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

Clause 33. A method for preparing a single stranded target DNA (ssDNA), comprising: (a) providing a template DNA comprising a target donor DNA sequence; (b) providing an amplification primer pair comprising a forward primer and a reverse primer designed to amplify the target donor DNA sequence, wherein the forward primer comprises a 5′ end comprising ribonucleotides and a 3′ end comprising deoxynucleotides, and wherein the reverse primer is not susceptible to digestion by RNase H; (c) amplifying the target donor DNA sequence with the forward and reverse primers to generate an amplification product comprising a first strand and a second strand complementary to the first strand, wherein the 5′ end of the first strand of the amplification product is susceptible to digestion by RNaseH; (d) contacting the amplification product with an RNaseH exonuclease, and; (e) contacting the amplification product with a second exonuclease that is a 5′ to 3′ exonuclease.

Clause 34. The method of clause 33, further comprising contacting the amplification product with a third exonuclease.

Clause 35. The method of clause 33 or 34, wherein the second exonuclease is Lambda exonuclease.

Clause 36. The method of any one of clauses 33 to 35, wherein the third exonuclease is a 3′ to 5′ exonuclease.

Clause 37. The method of clause 36, wherein the third exonuclease is Exonuclease III.

Clause 38. The method of any one of clauses 33 to 37, wherein (c) and (d) are simultaneous.

Clause 39. The method of any one of clauses 33 to 37, wherein (c) and (d) are sequential.

Clause 40. The method of any one of clauses 33 to 39, wherein the amplification product is contacted with the RNaseH, second exonuclease, and third exonuclease simultaneously.

Clause 41. The method of any one of clauses 33 to 40, wherein the amplification product is generated by polymerase chain reaction.

Clause 42. A method for genetically modifying a cell, the method comprising introducing into the cell: (i) at least one donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

Clause 43. A method for improving targeting efficiency of a nucleic acid cutting entity for genetic modification of a cell, comprising introducing into the cell: (i) at least one donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

Clause 44. A method for reducing off-target integration of a donor DNA during genetic modification of a cell, comprising introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

Clause 45. A method for enhancing delivery of a donor DNA to a cell for genetic modification of the cell, comprising introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

Clause 46. A method for reducing degradation of a donor DNA for genetic modification of a cell, comprising introducing into the cell: (i) the donor DNA molecule; (ii) at least one single stranded DNA binding protein or nucleic acid encoding the at least one single stranded DNA binding protein; and (iii) at least one nucleic acid cutting entity or nucleic acid encoding the at least one nucleic acid cutting entity; under conditions that allow for genetically modifying the cell at a predetermined locus.

Clause 47. A method for genetically modifying a cell, comprising introducing into the cell a donor DNA and at least one non-specific single strand DNA binding protein or nucleic acid encoding the at least one non-specific single stranded DNA binding protein in the presence of at least one nucleic acid cutting entity or nucleic acid encoding the at least one nucleic acid cutting entity; under conditions that allow for genetically modifying the cell at a predetermined locus.

Clause 48. The method of any one of clauses 42 to 47, wherein one or more of the at least one nucleic acid cutting entity is selected from a zinc finger nuclease; a TAL effector nuclease; and a CRISPR complex.

Clause 49. The method of clause 48, wherein the CRISPR complex is a Cas9/gRNA complex.

Clause 50. The method of any one of clauses 42 to 49, wherein the donor DNA molecule is contacted with the at least one single stranded DNA binding protein prior to introduction into the cell.

Clause 51. The method of any one of clauses 42 to 50, wherein the at least one nucleic acid cutting entity or nucleic acid encoding the at least one nucleic acid cutting entity is introduced into the cell before introduction of the donor DNA and/or at least one non-specific single strand DNA binding protein or nucleic acid encoding the at least one non-specific single stranded DNA binding protein into the cell.

Clause 52. The method of any one of clauses 42 to 50, wherein the at least one nucleic acid cutting entity or nucleic acid encoding the at least one nucleic acid cutting entity is introduced into the cell after introduction of the donor DNA and/or at least one non-specific single strand DNA binding protein or nucleic acid encoding the at least one non-specific single stranded DNA binding protein into the cell.

Clause 53. The method of any one of clauses 42 to 52, wherein the non-specific DNA binding protein comprises a oligonucleotide/oligosaccharide-binding (OB)-fold.

Clause 54. The method of any one of clauses 42 to 53, wherein the non-specific DNA binding protein is SSB, RecA, or T4G32.

Clause 55. The method of clause 54, wherein the DNA binding protein is SSB or a variant thereof.

Clause 56. The method of clause 55, wherein the SSB is E. coli SSB or variant thereof.

Clause 57. The method of any one of clauses 42 to 56, wherein one or more of the at least one non-specific DNA binding protein comprises the E. coli SSB C-terminus or variant thereof.

Clause 58. The method of any one of clauses 42 to 57, wherein the DNA binding protein is present in an amount sufficient to protect the donor DNA from degradation.

Clause 59. The method of any one of clauses 42 to 58, wherein the DNA binding protein is present in an amount sufficient to improve the targeting efficiency of the at least one nucleic acid cutting entity.

Clause 60. The method of any one of clauses 42 to 59, wherein the donor DNA comprises a nuclear localization signal (NLS).

Clause 61. The method of any one of clauses 42 to 60, wherein the DNA binding protein comprises a nuclear localization signal (NLS).

Clause 62. The method of any one of clauses 42 to 61, wherein the donor DNA and single-strand DNA binding protein are introduced into the cell using lipid transfection.

Clause 63. The method of any one of clauses 42 to 61, wherein the donor DNA and single-strand DNA binding protein are introduced into the cell using electroporation.

Clause 64. The method of any one of clauses 42 to 63, wherein the donor DNA is a single stranded DNA.

Clause 65. The method of any one of clauses 42 to 63, wherein the donor DNA is a double stranded DNA.

Clause 66. The method of any one of clauses 42 to 65, wherein the donor DNA is contacted with the DNA binding protein prior to introduction into the cell.

Clause 67. The method of any one of clauses 42 to 66, wherein the donor DNA is between 35 nucleotides and 10,000 nucleotides long.

Clause 68. A composition comprising cells, a donor DNA, at least one non-specific single strand DNA binding protein, and at least one nucleic acid cutting entity.

Clause 69. The composition of clause 68, wherein one or more of the at least one nucleic acid cutting entity is selected from a zinc finger nuclease; a TAL effector nuclease; and a CRISPR complex.

Clause 70. The composition of clause 69, wherein the CRISPR complex is a Cas9/gRNA complex.

Clause 71. The composition of any one of clauses 68 to 70, wherein one or more of the at least one non-specific DNA binding protein comprises an oligonucleotide/oligosaccharide-binding (OB)-fold.

Clause 72. The composition of any one of clauses 68 to 71, wherein one or more of the at least one non-specific DNA binding protein is SSB, RecA, or T4G32.

Clause 73. The composition of clause 72, wherein one or more of the at least one non-specific DNA binding protein is SSB or a variant thereof.

Clause 74. The composition of clause 73, wherein the SSB is E. coli SSB or variant thereof.

Clause 75. The composition of any one of clauses 68 to 74, wherein one or more of the at least one non-specific single strand DNA binding protein comprises the E. coli SSB C-terminus or variant thereof.

Clause 76. The composition of any one of clauses 68 to 75, wherein the donor DNA comprises a nuclear localization signal (NLS).

Clause 77. The composition of any one of clauses 68 to 76, wherein the DNA binding protein comprises a nuclear localization signal (NLS).

Clause 78. The composition of any one of clauses 68 to 77, wherein the donor DNA is a single stranded DNA.

Clause 79. The composition of any one of clauses 68 to 77, wherein the donor DNA is a double stranded DNA.

Clause 80. The composition of any one of clauses 68 to 79, wherein the donor DNA is between 35 nucleotides and 10,000 nucleotides long.

Clause 81. A kit for genetic modification, comprising (i) a non-specific single strand DNA binding protein or nucleic acid encoding the at least one non-specific single stranded DNA binding protein; and (ii) a nucleic acid cutting entity or a nucleic acid encoding the nucleic acid cutting entity.

Clause 82. The kit of clause 81, further comprising a lipid transfection reagent.

Clause 83. The kit of clause 81 or 82, further comprising a non-homologous end joining (NHEJ) inhibitor.

Clause 84. A method for preparing single stranded DNA (ssDNA), comprising denaturing a double stranded DNA (dsDNA) in the presence of a single-strand DNA binding protein, thereby preparing ssDNA.

Clause 85. The method of clause 84, wherein the single-strand DNA binding protein is SSB.

Clause 86. The method of clause 85, wherein the SSB is a thermostable SSB.

Clause 87. The method of any one of clauses 84 to 86, wherein the single strand DNA binding protein comprises the E. coli SSB C-terminus or variant thereof.

Clause 88. The method of any one of clauses 84 to 87, wherein one strand of the dsDNA is labeled.

Clause 89. The method of clause 88, further comprising isolating the labeled strand.

Clause 90. The method of clause 88, further comprising depleting the labeled strand.

Clause 91. A method for genetically modifying a cell, comprising introducing into the cell the ssDNA made by the method of any one of clauses 84 to 90 in the presence of a nucleic acid cutting entity or a nucleic acid encoding the nucleic acid cutting entity.

What is claimed is:

1. A method for preparing single stranded DNA (ssDNA), comprising:
(a) providing a composition comprising a double stranded DNA (dsDNA) having a modification on the 5′ end of one strand;
(b) contacting the composition with a lambda exonuclease; and
(c) contacting the composition comprising the lambda exonuclease with exonuclease III or T7 exonuclease;
wherein step (b) and step (c) are performed without changing buffer; thereby preparing ssDNA.

2. A method for preparing single stranded DNA (ssDNA), comprising:
(a) providing a composition comprising a double stranded DNA (dsDNA) having a modification on the 5′ end of one strand;
(b) contacting the composition with a lambda exonuclease; and
(c) contacting the composition comprising the lambda exonuclease with exonuclease III or T7 exonuclease;
wherein step (c) is performed without inactivating the lambda exonuclease; thereby preparing ssDNA.

3. The method of claim 1 or 2, wherein the 5′ modification is a 5′ phosphate.

4. The method of any one of claims 1 to 3, wherein the composition further comprises a detectable marker that binds to a nucleotide.

5. The method of claim 4, wherein the detectable marker is a fluorescent nucleic acid stain.

6. The method of claim 5, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

7. The method of any one of claims 4 to 6, further comprising monitoring an amount of detectable marker in the composition.

8. The method of claim 7, wherein the exonuclease III is added when the amount of the detectable marker reaches a plateau.

9. The method of any one of claims 1 to 8, wherein the temperature of the composition does not exceed about 50° C.

10. The method of claim 9, wherein the temperature of the composition is maintained between about 20° C. and about 50° C.

11. The method of claim 9, wherein the temperature of the composition is maintained between about 30° C. and about 40° C.

12. The method of claim 9, wherein the temperature of the composition is maintained at about 37° C.

13. The method of one of claims 1 to 12, further comprising adding a stop buffer.

14. The method of one of claims 1 to 13, further comprising purifying the ssDNA.

15. The method of any one of claims 1 to 14, wherein the modification comprises a phosphate.

16. The method of any one of claims 1 to 15, wherein the dsDNA is between 100 and 10,000 base pairs in length.

17. The method of any one of claims 1 to 16, wherein the composition comprises a magnesium salt.

18. The method of claim 17, wherein the composition comprises between 1 mM and 10 mM MgCl₂.

19. A composition comprising dsDNA, a lambda exonuclease, exonuclease III, and a detectable marker that binds to nucleotides.

20. The composition of claim 19, further comprising a magnesium salt.

21. The composition of claim 19 or 20, wherein the detectable marker is a fluorescent nucleic acid stain.

22. The composition of claim 21, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

23. A kit comprising a lambda exonuclease, exonuclease III, and a detectable marker that binds to nucleotides.

24. The kit of claim 23, further comprising a magnesium salt.

25. The kit of claim 23 or 24, wherein the detectable marker is a fluorescent nucleic acid stain.

26. The kit of claim 25, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

27. The kit of any one of claims 23 to 26, further comprising a reaction buffer, wherein the reaction buffer is compatible with lambda exonuclease and exonuclease III.

28. A system comprising:
(a) a device configured to detect a detectable marker that binds to a nucleotide; and
(b) a composition comprising DNA, an exonuclease, and a detectable marker that binds to nucleotides.

29. The system of claim 28, wherein the exonuclease comprises a lambda exonuclease and/or exonuclease III.

30. The system of claim 28, wherein the exonuclease comprises a lambda exonuclease and exonuclease III.

31. The system of any one of claims 28 to 30, wherein the detectable marker is a fluorescent nucleic acid stain.

32. The system of claim 31, wherein the fluorescent nucleic acid stain is (2-(n-bis-(3-dimethylaminopropyl)-amino)-4-(2,3-dihydro-3-methyl-(benzo-1,3-thiazol-2-yl)-methylidene)-1-phenyl-quinolinium.

33. A method for preparing a single stranded target DNA (ssDNA), comprising:
(a) providing a template DNA comprising a target donor DNA sequence;
(b) providing an amplification primer pair comprising a forward primer and a reverse primer designed to amplify the target donor DNA sequence, wherein the forward primer comprises a 5′ end comprising ribonucleotides and a 3′ end comprising deoxynucleotides, and wherein the reverse primer is not susceptible to digestion by RNase H;
(c) amplifying the target donor DNA sequence with the forward and reverse primers to generate an amplification product comprising a first strand and a second strand complementary to the first strand, wherein the 5′ end of the first strand of the amplification product is susceptible to digestion by RNaseH;
(d) contacting the amplification product with an RNaseH exonuclease, and;
(e) contacting the amplification product with a second exonuclease that is a 5′ to 3′ exonuclease.

34. The method of claim 33, further comprising contacting the amplification product with a third exonuclease.

35. The method of claim 33 or 34, wherein the second exonuclease is Lambda exonuclease.

36. The method of any one of claims 33 to 35, wherein the third exonuclease is a 3′ to 5′ exonuclease.

37. The method of claim 36, wherein the third exonuclease is Exonuclease III.

38. The method of any one of claims 33 to 37, wherein (c) and (d) are simultaneous.

39. The method of any one of claims 33 to 37, wherein (c) and (d) are sequential.

40. The method of any one of claims 33 to 39, wherein the amplification product is contacted with the RNaseH, second exonuclease, and third exonuclease simultaneously.

41. The method of any one of claims 33 to 40, wherein the amplification product is generated by polymerase chain reaction.

42. A method for genetically modifying a cell, the method comprising introducing into the cell:
(i) at least one donor DNA molecule;
(ii) at least one single stranded DNA binding protein or nucleic acid encoding the at least one single stranded DNA binding protein; and
(iii) at least one nucleic acid cutting entity or nucleic acid encoding the at least one nucleic acid cutting entity;
under conditions that allow for genetically modifying the cell at a predetermined locus.

43. A method for improving targeting efficiency of a nucleic acid cutting entity for genetic modification of a cell, comprising introducing into the cell:
(i) at least one donor DNA molecule;
(ii) at least one single stranded DNA binding protein or nucleic acid encoding the at least one single stranded DNA binding protein; and
(iii) at least one nucleic acid cutting entity or nucleic acid encoding the at least one nucleic acid cutting entity;
under conditions that allow for genetically modifying the cell at a predetermined locus.

44. A method for reducing off-target integration of a donor DNA during genetic modification of a cell, comprising introducing into the cell:
(i) the donor DNA molecule;
(ii) at least one single stranded DNA binding protein or nucleic acid encoding the at least one single stranded DNA binding protein; and
(iii) at least one nucleic acid cutting entity or nucleic acid encoding the at least one nucleic acid cutting entity;
under conditions that allow for genetically modifying the cell at a predetermined locus.

45. A method for enhancing delivery of a donor DNA to a cell for genetic modification of the cell, comprising introducing into the cell:
(i) the donor DNA molecule;
(ii) at least one single stranded DNA binding protein or nucleic acid encoding the at least one single stranded DNA binding protein; and
(iii) at least one nucleic acid cutting entity or nucleic acid encoding the at least one nucleic acid cutting entity;
under conditions that allow for genetically modifying the cell at a predetermined locus.

46. A method for reducing degradation of a donor DNA for genetic modification of a cell, comprising introducing into the cell:
(i) the donor DNA molecule;
(ii) at least one single stranded DNA binding protein or nucleic acid encoding the at least one single stranded DNA binding protein; and
(iii) at least one nucleic acid cutting entity or nucleic acid encoding the at least one nucleic acid cutting entity;
under conditions that allow for genetically modifying the cell at a predetermined locus.

47. A method for genetically modifying a cell, comprising introducing into the cell a donor DNA and at least one non-specific single strand DNA binding protein or nucleic acid encoding the at least one non-specific single stranded DNA binding protein in the presence of at least one nucleic acid cutting entity or nucleic acid encoding the at least one nucleic acid cutting entity; under conditions that allow for genetically modifying the cell at a predetermined locus.

48. The method of any one of claims 42 to 47, wherein one or more of the at least one nucleic acid cutting entity is selected from a zinc finger nuclease; a TAL effector nuclease; and a CRISPR complex.

49. The method of claim 48, wherein the CRISPR complex is a Cas9/gRNA complex.

50. The method of any one of claims 42 to 49, wherein the donor DNA molecule is contacted with the at least one single stranded DNA binding protein prior to introduction into the cell.

51. The method of any one of claims 42 to 50, wherein the at least one nucleic acid cutting entity or nucleic acid encoding the at least one nucleic acid cutting entity is introduced into the cell before introduction of the donor DNA and/or at least one non-specific single strand DNA binding protein or nucleic acid encoding the at least one non-specific single stranded DNA binding protein into the cell.

52. The method of any one of claims 42 to 50, wherein the at least one nucleic acid cutting entity or nucleic acid encoding the at least one nucleic acid cutting entity is introduced into the cell after introduction of the donor DNA and/or at least one non-specific single strand DNA binding protein or nucleic acid encoding the at least one non-specific single stranded DNA binding protein into the cell.

53. The method of any one of claims 42 to 52, wherein the non-specific DNA binding protein comprises a oligonucleotide/oligosaccharide-binding (OB)-fold.

54. The method of any one of claims 42 to 53, wherein the non-specific DNA binding protein is SSB, RecA, or T4G32.

55. The method of claim 54, wherein the DNA binding protein is SSB or a variant thereof.

56. The method of claim 55, wherein the SSB is E. coli SSB or variant thereof.

57. The method of any one of claims 42 to 56, wherein one or more of the at least one non-specific DNA binding protein comprises the E. coli SSB C-terminus or variant thereof.

58. The method of any one of claims 42 to 57, wherein the DNA binding protein is present in an amount sufficient to protect the donor DNA from degradation.

59. The method of any one of claims 42 to 58, wherein the DNA binding protein is present in an amount sufficient to improve the targeting efficiency of the at least one nucleic acid cutting entity.

60. The method of any one of claims 42 to 59, wherein the donor DNA comprises a nuclear localization signal (NLS).

61. The method of any one of claims 42 to 60, wherein the DNA binding protein comprises a nuclear localization signal (NLS).

62. The method of any one of claims 42 to 61, wherein the donor DNA and single-strand DNA binding protein are introduced into the cell using lipid transfection.

63. The method of any one of claims 42 to 61, wherein the donor DNA and single-strand DNA binding protein are introduced into the cell using electroporation.

64. The method of any one of claims 42 to 63, wherein the donor DNA is a single stranded DNA.

65. The method of any one of claims 42 to 63, wherein the donor DNA is a double stranded DNA.

66. The method of any one of claims 42 to 65, wherein the donor DNA is contacted with the DNA binding protein prior to introduction into the cell.

67. The method of any one of claims 42 to 66, wherein the donor DNA is between 35 nucleotides and 10,000 nucleotides long.

68. A composition comprising cells, a donor DNA, at least one non-specific single strand DNA binding protein, and at least one nucleic acid cutting entity.

69. The composition of claim 68, wherein one or more of the at least one nucleic acid cutting entity is selected from a zinc finger nuclease; a TAL effector nuclease; and a CRISPR complex.

70. The composition of claim 69, wherein the CRISPR complex is a Cas9/gRNA complex.

71. The composition of any one of claims 68 to 70, wherein one or more of the at least one non-specific DNA binding protein comprises an oligonucleotide/oligosaccharide-binding (OB)-fold.

72. The composition of any one of claims 68 to 71, wherein one or more of the at least one non-specific DNA binding protein is SSB, RecA, or T4G32.

73. The composition of claim 72, wherein one or more of the at least one non-specific DNA binding protein is SSB or a variant thereof.

74. The composition of claim 73, wherein the SSB is E. coli SSB or variant thereof.

75. The composition of any one of claims 68 to 74, wherein one or more of the at least one non-specific single strand DNA binding protein comprises the E. coli SSB C-terminus or variant thereof.

76. The composition of any one of claims 68 to 75, wherein the donor DNA comprises a nuclear localization signal (NLS).

77. The composition of any one of claims 68 to 76, wherein the DNA binding protein comprises a nuclear localization signal (NLS).

78. The composition of any one of claims 68 to 77, wherein the donor DNA is a single stranded DNA.

79. The composition of any one of claims 68 to 77, wherein the donor DNA is a double stranded DNA.

80. The composition of any one of claims 68 to 79, wherein the donor DNA is between 35 nucleotides and 10,000 nucleotides long.

81. A kit for genetic modification, comprising (i) a non-specific single strand DNA binding protein or nucleic acid encoding the at least one non-specific single stranded DNA binding protein; and (ii) a nucleic acid cutting entity or a nucleic acid encoding the nucleic acid cutting entity.

82. The kit of claim 81, further comprising a lipid transfection reagent.

83. The kit of claim 81 or 82, further comprising a non-homologous end joining (NHEJ) inhibitor.

84. A method for preparing single stranded DNA (ssDNA), comprising denaturing a double stranded DNA (dsDNA) in the presence of a single-strand DNA binding protein, thereby preparing ssDNA.

85. The method of claim 84, wherein the single-strand DNA binding protein is SSB.

86. The method of claim 85, wherein the SSB is a thermostable SSB.

87. The method of any one of claims 84 to 86, wherein the single strand DNA binding protein comprises the E. coli SSB C-terminus or variant thereof.

88. The method of any one of claims 84 to 87, wherein one strand of the dsDNA is labeled.

89. The method of claim 88, further comprising isolating the labeled strand.

90. The method of claim 88, further comprising depleting the labeled strand.

91. A method for genetically modifying a cell, comprising introducing into the cell the ssDNA made by the method of any one of claims 84 to 90 in the presence of a nucleic acid cutting entity or a nucleic acid encoding the nucleic acid cutting entity.


SEQUENCE LISTING (20 sequences)

SEQ ID NO: 1 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 2 - protein, 174 aa, Citrobacter sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGSNQFSGGAQSRPQQSAPAAPSNEPPMDFDDDIPF

SEQ ID NO: 3 - protein, 174 aa, Citrobacter sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGTPAGGGQQQGGWGQPQQPQGGNQFSGGAHSRPQQSAPAAPSNEPPMDFDDDIPF

SEQ ID NO: 4 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGSNIGGGQPQGGWGQPQQPQGGNQFSGGAQSRPQQSAPAAPSNEPPMDFDDDIPF

SEQ ID NO: 5 - protein, 267 aa, Thermus aquaticus
MARGLNQVFLIGTLTARPDMRYTPGGLAILDLNLAGQDAFTDESGQEREVPWYHRVRLLGRQAEMWGDLLEKGQLIFVEGRLEYRQWEKDGEKKSEVQVRAEFIDPLEGRGRETLEDARGQPRLRRALNQVILMGNLTRDPDLRYTPQGTAVVRLGLAVNERRRGQEAVNAERTHFLEVQAWRELAEWASELRKGDGLLVIGRLVNDSWTSSSGERRFQTRVEALRLERPTRGPAQAGGSRPPTVQTGGVDIDEGLEDFPPEEDLPF

SEQ ID NO: 6 - protein, 266 aa, Thermus brockianus
MARGLNRVYLIGSLTSRPDMRYTPGGLAILELNLAGQDTLWDESGQERELPWYHRVRLLGRQAEMWGDVLEKGQLLFVEGRLEYRQWERDGEKRSELQVRADFIDPLDARGRETQEDAKSQPRLRHALNQVVLMGNLTRDAELRYTPQGTAVARLGLAVNERRRGPGAEEEKTHFIEVQAWRELAEWAGELRKGDGLLVIGRLVNDSWTSSSGERRFQTRVEALRLERPTRGPAQTGGSRPQPVQTGGVDIDEGLEDFPPEEDLPF

SEQ ID NO: 7 - protein, 264 aa, Thermus oshimai
MARGLNRVFLIGTLTARPDMRYTPSGMAILELNLAGQDLIRDETGQERELPWYHRVRLFGRQAEMWGDVLEKGQLLFVEGRLSYRQWEREGEKRSQLEIRADFIDPLAPAGRETLEDTRGQPRLKNALNQVYLMGNLTRDPELRYTPQGTAVARLGLAVNERGRQQGEERTHFVEVQAWRDLAEWAGELRKGEGLFVIGRLVNDSWTSSTGERRYQTRVEALRLERPTRGPAQAGGSRPQTVQTGGVDIDEGLEDFPPEEDLPF

SEQ ID NO: 8 - DNA, 97 bp, Artificial Sequence
  source: /note=“Description of Artificial Sequence Synthetic oligonucleotide”
cggggtagcgggcgaagcactgcacgccgtaggtgaaggtggtcacgagggtgggccagggcacgggcagcttgccggtggtgcagatgaacttcag

SEQ ID NO: 9 - DNA, 1486 bp, Artificial Sequence
  source: /note=“Description of Artificial Sequence Synthetic polynucleotide”
cacagcgcgcccggctattctcgcagctcaccatgggaggtaagcccttgcattcgaccgagtacaagcccacagtgcggctggccaccagggacgatgtgcctagagctgtgcggacactggccgctgccttcgccgattaccctgccaccagacacaccgtggaccccgacagacacatcgagagagtgaccgagctgcaggaactgtttctgaccagagtgggcctggacatcggcaaagtgtgggtggccgatgatggcgccgctgtggctgtgtggacaacccctgagtctgtggaagccggcgctgtgttcgccgagatcggacctagaatggccgagctgagcggctctagactggctgcccagcagcagatggaaggcctgctggccccccacagacctaaagagcctgcctggtttctggccaccgtgggcgtgtcacctgaccaccagggcaagggactgggatctgctgtggtgctgcctggcgtggaagctgctgaaagggctggcgtgcccgccttcctggaaacaagcgcccccagaaacctgcccttctacgagagactgggcttcaccgtgaccgccgacgtggaagtgcctgagggccctagaacctggtgcatgaccagaaagcctggcgccggaagcggagctactaacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggttcaggtagtggaagcggtgatgatgatatcgccgcgctcgtcgtcgacaacgg

SEQ ID NO: 10 - DNA, 720 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 11 - DNA, 720 bp, Artificial Sequence; identical to SEQ ID NO: 10
  source: /note=“Description of Artificial Sequence Synthetic polynucleotide”
  misc_feature (193)..(198): /note=“May or may not be present”

SEQ ID NO: 12 - protein, 301 aa, Escherichia virus T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 13 - protein, 356 aa, Escherichia virus T4
MSVLEKLKKNSTLKTTAVLSKSSFFNEKTNTRTKIPMLNIAFSGDLKKGFQSGLIFFAGPSKHFKSNMGLTCVSAYMKQNPDAACLFFDSEFGITPAYLESMGVDPDRVVHVPIKNIEELKFEIMNQLEQITREDKVIIFIDSIGNLASKKEVEDAINEKSAQDMTRAKALKGLFRMVTPYLTMNDIPCIAINHTYETQEMFSKTVMSGGTGAMYSANEVFIIGRRQQKEGTEITGYDFILNAEKSRTVKEKSKFPISVTFSGGIDPYSGLLELAVELGWVVKPSNGWYSRSILNTETGEMETEERKFRAKETNSIEFWKPLLTNDKFNEAINDHYKLGQVISDEAVDKEIEDMLA

SEQ ID NO: 14 - protein, 135 aa, Escherichia virus T4
MDLNDLKEQLEADMKIDATKLQWEALNNPVVYSKWLRIYSEAKRETIALEAKKKKAMKNRLDFYTNRSDDWCRAEYEKSELKVVMAADDEILPLDTKIAYYQMVMDFAGRALDIVKSRGFAIKNAIELRMLESGR

SEQ ID NO: 15 - protein, 1417 aa, Unknown
  source: /note=“Description of Unknown Bloom syndrome protein sequence”
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 16 - protein, 339 aa, Unknown
  source: /note=“Description of Unknown Rad51 Protein sequence”
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 17 - protein, 765 aa, Unknown
  source: /note=“Description of Unknown Topoisomerase I sequence”
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 18 - protein, 314 aa, Vaccinia virus
MRALFYKDGKLFTDNNFLNPVLDDNPAYEVLQHVKIPTHLTDVVVYEQTWEEALTRLIFVGSDSKGRRQYFYGKMHVQNRNAKRDRIFVRVYNVMKRINCFINKNIKKSSTDSNYQLAVFMLMETMFFIRFGKMKYLKENETVGLLTLKNKHIEISPDEIVIKFVGKDKVSHEFVVHKSNRLYKPLLKLTDDSSPEEFLFNKLSERKVYECIKQFGIRIKDLRTYGVNYTFLYNFWTNVKSISPLPSPKKLIALTIKQTAEVVGHTPSISKRAYMATTILEMVKDKNFLDVVSKTTFDEFLSIVVDHVKSSTDG

SEQ ID NO: 19 - protein, 177 aa, Artificial Sequence
  source: /note=“Description of Artificial Sequence Synthetic polypeptide”
MASRGVNKVILVGNLGQDPEVRYMPNGGAVANITLATSESWRDKATGEMKEQTEWHRVVLFGKLAEVASEYLRKGSQVYIEGQLRTRKWTDQSGVDKYTTEVVVNVGGTMQMLGGRQGGGAPAGNIGGGQQQGGWGQPQQPQGGNQFSGGAQSRPQQSAPAAPSNEPPMDFDDDIPF

SEQ ID NO: 20 - protein, 261 aa, Artificial Sequence
  source: /note=“Description of Artificial Sequence Synthetic polypeptide”
MARGLNRVFLIGTLTARPDMRYTPGGLAILELNLAGQDIDESGQERELPWYHRVRLLGRQAEMWGDVLEKGQLLFVEGRLEYRQWERDGEKRSELQVRADFIDPLDARGRETLEDARGQPRLRALNQVILMGNLTRDPELRYTPQGTAVARLGLAVNERRRGQAEERTHFIEVQAWRELAEWAGELRKGDGLLVIGRLVNDSWTSSSGERRFQTRVEALRLERPTRGPAQAGGSRPQTVQTGGVDIDEGLEDFPPEEDLPF